SEQUENCE LISTING (12 sequences)

SEQ ID NO: 1 - DNA, 3623 bp, Brassica napus
  source 1..3623: mol_type = unassigned DNA
atggctctctcattagcttcttctccttcctcttgtcgcacttggttgtacgatgttttccctagcttcagtggggtagacgttcgtgttactttcctcagccacttgttgaaggagtttgacaaaaagttgatcactgctttcaaagacaacgagatcgagagaagtcgatcactggatcccgagcttaaacaagccattaaagattcgaggatcgcagtggttatcttctcccaaaactatgcctcttcaagctggtgtcttaatgagttgttagagatagtcaagtgtggtcaaatggtgatacctgttttctaccggttggatccttcccatgtgaggaaacaaaccggtgactttggtaagttctttgaagaaacatgcaacaacaaaacagaggaagagaaaatacagtggaggagagctttgaccgatgtagccaatactctcgggtatcattcagtaaactggggtaacgaagctgcaatgattgaagaaatcgccaatgatgttttggataaactacttttaacttcatcgaaggattcagagaactttgtgggcatcgaagatcatgttgcaaaactgagtgtattgctgcagttggacgcggaggaagtgaggatggttggtttatggggttcctcagggatcggcaagactacaattgcaagagttctgtttcaacgactttctcgacacttccgaggtagcattttcatagacagggctttcgtatctaagactatggaaattttcaaggcagctaatccggacgactataacatgaagctgcatttgcaaagaaatttcctatctgaaatcttaggtaaaggagacataaagataaatcatttgagtgcagttggggagaggctgaagaatcagaaagttcttattttcattgatgattttgatgatcaagttgtgctagaagccttggttggtcaaactcaatggtttggaagtgggagcagaatcgttgtggttacaaatgataagcagtatctaagggcccatgggattaatcacatttacaaggtctgtctcccaactaaaaagctagctgttgagatgttatgtcgatctgctttcaggaaaaaggctgcacctgaaggttttgaggagcttgtagctaaagttacaggacttgctggtagtcttcctttaggtcttaatgttttgggttcatatctacggggaagggacaaggagtactggatggatttgttgccaaggcttcagaatggtttagatgggaaaattgagaagacattgagagtcagctacgatggattaacaagcgaagaagataaagcgttatttcgccatattgcatgccttttccagtgggaaaaagtcacatacctgaagttgctgctcgctgatagtgggttgagtgttacggttgggctggaaaacctagctgataagtccctcattcatgtaagagaggattatgtgaagatgcaccgtttgttagaagagatgggtagacgtattgttaggcttgacgagcctgaaaaacgagaatttctggtggacgcacaagatatctgtgatgtactcagtcaagacactggtactcataagatattgggtataaaattgaatattgatgagattgatgaactgaatgtgcatgagaatgccttcaaagggatgcgcaatctgcgtttcctggaaattcactcacaaaaccgtcatgagtttggaaacgaagaagttagaattcacttacctgaaaacttcgactatttgcctcctaaacttaaaatattggattggtatgaatatccaatgagatgtctgccttctaagtttcgtcctgaaaaactcgtcaagctcaaaatggtgaatagcaagctcgagaagctgtgggaagggattgtgtcgcttacatgtcttaaaaagatgaatatgtcgggatctcaaaacttgatagaaatgccagatctttcaaaggccaccaatctggagacactatatcttgaggattgctttagtttggtcaagcttccttcctctattccacaccccaacaaactgacgacattaatcttgaagaactgtcgaaatgtggagactattccaattggcattagcctcaaatctcttaaaaacctacgtactgatggttgctcacggatgaggacttttccccaaatctcaagcaccatcgaagatgtctacataggcgcaacatccattgaagaaataccttcaaatttgagtttgtgtttcgagaatctccatacctttacgatgcacagcccaaagaaactatgggaaagagtgcagcttcttactctcctcacgacgatcatgtctccctctttgtggtatctggatctctcggataaccctggcttggtggagcttccttcttcatttaagaatctccataacctgaggagattggaaattagaaactgcgtaaatctggaaactcttcccaccggaatcaacctcggatctctcaaaatcctagatctcaggggatgctcacggttgaggacttttcctgatatctcaacccacatcacacatctttatctaagcggaacagggattgaagagattccttgttcgattgagaaattctccaggcttggctccctacatatgaacggatgcaacaatttggaatatgtaaacctaaacctttttaaactcaaacatcttcacgaagtcgacttttcagactgcaagtgcttaaacttggatcaagaagctctgtttcaaaagaaaacatattcagtttgtcaactgaagttgtcaggtgaagaagtgccttcatatttcacgcaccgtactactggaacctcctcctctctcaccattcctctacttcacagctgtatctcacaatcattcctccgattcagggcttgtattgtgtttgattcggacaaggacaatgagtcatatagcagatgtgcctttagattcaaaggcagttttcggaactgctctgattcctataatcaggcacaagacttctgcgcagtcacggatgattataagatccgttcatataagaaggatggttgtctgcttgtattagactaccagatgtctcaaatccctttagaaatgaacttcgatggcctggatctgaagattcatattgattattgtcgttctgctaaaataaaaggatggggtatacgaatcttagaggaggactgttcatcggcagacaaccgacttggttatccaaacattctaccacatgtttttgaagccgatgaatgcaatgaggctgatttcgtgaataaacttttgattgtgttaggttgtaacgcagagcatctaaaattaggaattaagagagattcccatccaacttgcttgtggatcttagtatgcggagaagcagtgcctctttttctatcagagattgctcctgcacagctcaggggaggtctcaacattgtattccaactcatggtcacaattggaatcctaatagccaaccttgtcaactacttcactgccaccgttcaccctaacggatggcgaatcgccctcggtggagccgcaatccccaccgttatcctagtcttcggttcactgatcatctgcgagactcccacgagcttcatagagcgcaagtgttg

SEQ ID NO: 2 - DNA, 4749 bp, Brassica napus
  source 1..4749: mol_type = genomic DNA
gccagctatttaggtgacactatagaatactcaagctatgcatcaagcttggtaccgagctcggatccactagtaacggccgccagtgtgctggaattcgcccttttgcgctacactccaatctgacaatttatatggactaacattcttcagaagatcaatcaccaagtccttcttttccttcatattatttttgctcctatctctatggctctctcattagcttcttctccttcctcttgtcgcacttggttgtacgatgttttccctagcttcagtggggtagacgttcgtgttactttcctcagccacttgttgaaggagtttgacaaaaagttgatcactgctttcaaagacaacgagatcgagagaagtcgatcactggatcccgagcttaaacaagccattaaagattcgaggatcgcagtggttatcttctcccaaaactatgcctcttcaagctggtgtcttaatgagttgttagagatagtcaagtgtggtcaaatggtgatacctgttttctaccggttggatccttcccatgtgaggaaacaaaccggtgactttggtaagttctttgaagaaacatgcaacaacaaaacagaggaagagaaaatacagtggaggagagctttgaccgatgtagccaatactctcgggtatcattcagtaaactggtacggttttatgcttcttttgaataaaggttagacttctggttgctagggggtgatattatctttcttattgttaggggtaacgaagctgcaatgattgaagaaatcgccaatgatgttttggataaactacttttaacttcatcgaaggattcagagaactttgtgggcatcgaagatcatgttgcaaaactgagtgtattgctgcagttggacgcggaggaagtgaggatggttggtttatggggttcctcagggatcggcaagactacaattgcaagagttctgtttcaacgactttctcgacacttccgaggtagcattttcatagacagggctttcgtatctaagactatggaaattttcaaggcagctaatccggacgactataacatgaagctgcatttgcaaagaaatttcctatctgaaatcttaggtaaaggagacataaagataaatcatttgagtgcagttggggagaggctgaagaatcagaaagttcttattttcattgatgattttgatgatcaagttgtgctagaagccttggttggtcaaactcaatggtttggaagtgggagcagaatcgttgtggttacaaatgataagcagtatctaagggcccatgggattaatcacatttacaaggtctgtctcccaactaaaaagctagctgttgagatgttatgtcgatctgctttcaggaaaaaggctgcacctgaaggttttgaggagcttgtagctaaagttacaggacttgctggtagtcttcctttaggtcttaatgttttgggttcatatctacggggaagggacaaggagtactggatggatttgttgccaaggcttcagaatggtttagatgggaaaattgagaagacattgagagtcagctacgatggattaacaagcgaagaagataaagcgttatttcgccatattgcatgccttttccagtgggaaaaagtcacatacctgaagttgctgctcgctgatagtgggttgagtgttacggttgggctggaaaacctagctgataagtccctcattcatgtaagagaggattatgtgaagatgcaccgtttgttagaagagatgggtagacgtattgttaggcttgacgagcctgaaaaacgagaatttctggtggacgcacaagatatctgtgatgtactcagtcaagacactgtaagttatctcttatgttcgtgctcctttcagtcaataaataagatttagagcatgccattttataagcaaaactaatacttgatattatataattttcagggtactcataagatattgggtataaaattgaatattgatgagattgatgaactgaatgtgcatgagaatgccttcaaagggatgcgcaatctgcgtttcctggaaattcactcacaaaaccgtcatgagtttggaaacgaagaagttagaattcacttacctgaaaacttcgactatttgcctcctaaacttaaaatattggattggtatgaatatccaatgagatgtctgccttctaagtttcgtcctgaaaaactcgtcaagctcaaaatggtgaatagcaagctcgagaagctgtgggaagggattgtggtaagttttgagaatagtttgtgatgttatttgtagtaagactaatctttattttattttttggatgacaatcttgttctactgagctcatgtgttctgttcctttttttttttgttgagtacagtcgcttacatgtcttaaaaagatgaatatgtcgggatctcaaaacttgatagaaatgccagatctttcaaaggccaccaatctggagacactatatcttgaggattgctttagtttggtcaagcttccttcctctattccacaccccaacaaactgacgacattaatcttgaagaactgtcgaaatgtggagactattccaattggcattagcctcaaatctcttaaaaacctacgtactgatggttgctcacggatgaggacttttccccaaatctcaagcaccatcgaagatgtctacataggcgcaacatccattgaagaaataccttcaaatttgagtttgtgtttcgagaatctccatacctttacgatgcacagcccaaagaaactatgggaaagagtgcaggtatgtatgtagttccaaactttgtgtgtttctccaatctgttttacgttatagatattagataatggtgcaaatgaaactaaggttatattgtatttatcggagggaagaagagtagcgctgaatatgattttgtgtatttggttcagcttcttactctcctcacgacgatcatgtctccctctttgtggtatctggatctctcggataaccctggcttggtggagcttccttcttcatttaagaatctccataacctgaggagattggaaattagaaactgcgtaaatctggaaactcttcccaccggaatcaacctcggatctctcaaaatcctagatctcaggggatgctcacggttgaggacttttcctgatatctcaacccacatcacacatctttatctaagcggaacagggattgaagagattccttgttcgattgagaaattctccaggcttggctccctacatatgaacggatgcaacaatttggaatatgtaaacctaaacctttttaaactcaaacatcttcacgaagtcgacttttcagactgcaagtgcttaaacttggatcaagaagctctgtttcaaaagaaaacatattcagtttgtcaactgaagttgtcaggtgaagaagtgccttcatatttcacgcaccgtactactggaacctcctcctctctcaccattcctctacttcacagctgtatctcacaatcattcctccgattcagggcttgtattgtgtttgattcggacaaggacaatgagtcatatagcagatgtgcctttagattcaaaggcagttttcggaactgctctgattcctataatcaggcacaagacttctgcgcagtcacggatgattataagatccgttcatataagaaggatggttgtctgcttgtattagactaccagatgtctcaaatccctttagaaatgaacttcgatggcctggatctgaagattcatattgattattgtcgttctgctaaaataaaaggatggggtatacgaatcttagaggaggactgttcatcggcagacaaccgacttggttatccaaacattctaccacatgtttttgaagccgatgaatgcaatgaggctggtgaatgtgggaggcaaatgatgtagtgacggaaagaagcgggtaaaggcattaatcatgaacttatcacagtattttctatatcataatttcttgtactgagacttttttttctttttctgcagatttcgtgaataaacttttgattgtgttaggttgtaacgcagagcatctaaaattaggaattaagagagattcccatccaacttgcttgtggatcttagtatgcggagaagtaagagcacaaaattgtgggaaggagtagtgcatgtatgttttcttcaaccttccttcctttatactagttcttacaaaaatggttatgcctggcagatctctagaaatgacctaacattattgtgtgtaccttaaattctcaggcagtgcctctttttctatcagagattgctcctgcacagctcaggggaggtctcaacattgtattccaactcatggtcacaattggaatcctaatagccaaccttgtcaactacttcactgccaccgttcaccctaacggatggcgaatcgccctcggtggagccgcaatccccaccgttatcctagtcttcggttcactgatcatctgcgagactcccacgagcttcatagagcgcaagtgttgaaaccagcgagtcgtccacctttaatcattggaatgcttctacagcttttccagcagtttagtggaatcaatgctattatgttctacgcaccggttttaagggcgaattctgcagatatccatcacactggcggccgctcgagcatgcatctagagggcccaattcgccctatagtgagtcgtatacaatcactgc

SEQ ID NO: 3 - protein, 1207 aa, Brassica napus
MALSLASSPSSCRTWLYDVFPSFSGVDVRVTFLSHLLKEFDKKLITAFKDNEIERSRSLDPELKQAIKDSRIAVVIFSQNYASSSWCLNELLEIVKCGQMVIPVFYRLDPSHVRKQTGDFGKFFEETCNNKTEEEKIQWRRALTDVANTLGYHSVNWGNEAAMIEEIANDVLDKLLLTSSKDSENFVGIEDHVAKLSVLLQLDAEEVRMVGLWGSSGIGKTTIARVLFQRLSRHFRGSIFIDRAFVSKTMEIFKAANPDDYNMKLHLQRNFLSEILGKGDIKINHLSAVGERLKNQKVLIFIDDFDDQVVLEALVGQTQWFGSGSRIVVVTNDKQYLRAHGINHIYKVCLPTKKLAVEMLCRSAFRKKAAPEGFEELVAKVTGLAGSLPLGLNVLGSYLRGRDKEYWMDLLPRLQNGLDGKIEKTLRVSYDGLTSEEDKALFRHIACLFQWEKVTYLKLLLADSGLSVTVGLENLADKSLIHVREDYVKMHRLLEEMGRRIVRLDEPEKREFLVDAQDICDVLSQDTGTHKILGIKLNIDEIDELNVHENAFKGMRNLRFLEIHSQNRHEFGNEEVRIHLPENFDYLPPKLKILDWYEYPMRCLPSKFRPEKLVKLKMVNSKLEKLWEGIVSLTCLKKMNMSGSQNLIEMPDLSKATNLETLYLEDCFSLVKLPSSIPHPNKLTTLILKNCRNVETIPIGISLKSLKNLRTDGCSRMRTFPQISSTIEDVYIGATSIEEIPSNLSLCFENLHTFTMHSPKKLWERVQLLTLLTTIMSPSLWYLDLSDNPGLVELPSSFKNLHNLRRLEIRNCVNLETLPTGINLGSLKILDLRGCSRLRTFPDISTHITHLYLSGTGIEEIPCSIEKFSRLGSLHMNGCNNLEYVNLNLFKLKHLHEVDFSDCKCLNLDQEALFQKKTYSVCQLKLSGEEVPSYFTHRTTGTSSSLTIPLLHSCISQSFLRFRACIVFDSDKDNESYSRCAFRFKGSFRNCSDSYNQAQDFCAVTDDYKIRSYKKDGCLLVLDYQMSQIPLEMNFDGLDLKIHIDYCRSAKIKGWGIRILEEDCSSADNRLGYPNILPHVFEADECNEADFVNKLLIVLGCNAEHLKLGIKRDSHPTCLWILVCGEAVPLFLSEIAPAQLRGGLNIVFQLMVTIGILIANLVNYFTATVHPNGWRIALGGAAIPTVILVFGSLIICETPTSFIERKC

SEQ ID NO: 4 - DNA, 3729 bp, Brassica napus
  source 1..3729: mol_type = unassigned DNA
atggctctctcattagcttcttctccttcctcttctcgcacttggttgtacgatgttttccctagcttcagtggggtagacgtccgtgttactttcctcagccacttgttgaaggagtttgacaaaaagttgatcactgctttcaaagacaacgagatcgagagaagtcgatcactggatcccgagcttaaacaagccattaaagattcgaggatcgcggtggttatcttctcccaaaactatgcctcttcaagctggtgtcttaatgagttgttagagatagtcaagcgtggtcaaatggtgatacctgttttctaccggttggatccttcccacgtgaggaaacaaaccggtgacttcgggaaaatatttgaagaaacatgcaagaatcaaaaagaggaagtgataataattcaatggaggagagctttgaccgatgtagccaatacactcgggtatcattcagtaaactggggtaacgaagctgcaatgattgaagaaatcgccaatgatgttttggataaactacttttaacttcatcgaaggattcagagaactttgtgggcatcgaagatcatatcgcagaactgagtgtactgttgcagttggacgcggaggaagtgaggatggttggtttatggggttcctcagggatcggcaagactacaattgcaagagttctgtttcaacgactttctcgacacttccgaggtagcattttcatagacagggctttcgtatctaagactatggaaattttcaaggcagctaatccggacgactataacatgaagctgcatttgcaaagaaatttcctatctgaaatcttaggtaaaggagacataaagataaatcatttgagtgcagttggggagaggctgaagaatcagaaagttcttattttcattgatgattttgatgatcaagttgtgctagaagccttggttggtcaaactcaatggtttggaagtgggagcagaatcgttgtggttacaaatgataagcagtatctaagggcccatgggattaatcacatttacgaggtctgtctcccaactggaaacctagctgttgagatgttatgtcgatatgctttcaggaaaaaggctgcacctgaaggttttgaggagcttgtagctaaagttacaggacttgctggtagtcttcctttaggtcttaatgttttgggttcatctctacggggaagggacaaggagtactggatggatttgttgccaaggcttcggaattgtttagatgggaaaattgagaagacattgagagtcagctacgatggattaacaagcgaagaagataaagccttatttcgccatatcgcatgccttttcaatggtgcaacagtcacatacctgaagttggtgctcactgatagtgggttgagtgttaatgtggggctggaaaacctagctgataagtccctcattcatgaaagagaggattatgtggagatgcaccgtttgttagaagagatgggtagacgtattgttaggcttgaggagcctgaaaaacgagaatttctggtggacgcacaagatatctgtgatgtactcagtcaagacactggtactcataagatattgggtataaaattgaatattgatgagattgatgaactgaatgtgcatgagaatgccttcaaagggatgcgcaatctgcgtttcctggaaattcactcaaaaaagcgttatgtgtttggaaaggaagaagtaccaattcacttacctgaaaacttcgactatttgcctccaaaacttaaaatattggattggtatgaatatccaatgagatgtctgccttctaagtttcgtcctgaaaaactcgtcaagctcaaaatggtgaatagcaagctcgagaagctgtggaaagggattgtgtcgcttacatgtcttaataagatggatatgtcggcatctcaaaacttgatagaaatgccagatctttcaaaggccaccaatctggagacacttaaacttcggaattgctatagtttggtcaagcttccttcctctattccacatcccaacaaactgacgacattaaacttgaagaactgtcgaaatctggagactattccaattggcattagcctcaaatctctcaaaaacctaaatactaaaggatgctcacggatgaggatttttccccaaatctcaaccagcatcgtagatgtcgacatagccgcaacatccattgaagaaataccttcaaatttgagtttgtgtttcgagagtctccatacctttacgatgcacagcccaaagaaactatgggaaagagtgcagcttcttactctcctcacgacgatcatgtctccctctttgtggtatctggatctctcggataacccaggcttggtggagcttccttcttcatttaagaatctccataacttgcagatattgaaaattagtaactgcgtaaatctggaaactcttcccaccggaatcaacctcggatctctctggcaactagatctcagtggatgctcaaggttgaggacttttcctgatatctcaaccaacatcatagttctcgatctcagcgaaacagccatcgaagagattccttgttggattgagaaattctctaaccttaactcccttaggatgaagggatgcaacaatttggaatatgtaaacctaaacatttctaaactcaaacatcttcagaaagtagacttttcagactgcaagtcattgactggagctagcttgaataatcgtccaagagaaagtgccttgagttattaccacatatgctacattggtatcgatttcaccaagtgcttaaacttggatcaagaagctctgtttcaaaagaaaacatattttggttgtcatctgaagttgtcaggtgaagaagtgccttcatatttcactcaccgtactactggaacctcctcctctctcaccattcctttacttcacagctgtctctcacaaccattcctcctattcagggcttgtattgtgtttgattcggacaaggagacatatagcgattgtgtctttagattcaaaggcagttttcggaactgctctgattcctataatcaggcacaagacttctgcgcagtcacggatgattatgagatcaattcatatgagaaggatggttgtctgtttgtattagactaccagatgtctcaaatccctttagaaatgaacttcgatcgcctggatctgaagattcatattgttgattgttttaatgatgctaaaataaaaggatggggtatacgaatcttagaggaggactgttcatcggcagacaaccgacttggttatccaaacattctaccacatgtttttgaagccgatgaatgcaatatgaggctggtgaatgtggaggcaaatgatgcagtgacggaaagaagcggtatgcggagaagtaagagcacaatattgtgggaaggagtagtgcatgcagtgcctctttttctatcagagattgctcctgcacagctcaggggaggtctcaacattgtattccaactcatggtaacaattggaatcctaatagccaaccttgtcaactacttcactgccaccgttcaccctaacggatggcgaatcgccctcggtggagctgcaatccccgcggttatcctcctcttgggttcactgatcatctgtgagacccccacgagcctcatagagcgcaacaaaaacgaagaaggcagagaaactctaaggaaaatcagagga

SEQ ID NO: 5 - DNA, 4492 bp, Brassica napus
  source 1..4492: mol_type = genomic DNA
atggctctctcattagcttcttctccttcctcttctcgcacttggttgtacgatgttttccctagcttcagtggggtagacgtccgtgttactttcctcagccacttgttgaaggagtttgacaaaaagttgatcactgctttcaaagacaacgagatcgagagaagtcgatcactggatcccgagcttaaacaagccattaaagattcgaggatcgcggtggttatcttctcccaaaactatgcctcttcaagctggtgtcttaatgagttgttagagatagtcaagcgtggtcaaatggtgatacctgttttctaccggttggatccttcccacgtgaggaaacaaaccggtgacttcgggaaaatatttgaagaaacatgcaagaatcaaaaagaggaagtgataataattcaatggaggagagctttgaccgatgtagccaatacactcgggtatcattcagtaaactggtacggttttatgcatcattcattcttttgagtaaaggttagacttctggttgctagggggtgatattatatttcttaatgttaggggtaacgaagctgcaatgattgaagaaatcgccaatgatgttttggataaactacttttaacttcatcgaaggattcagagaactttgtgggcatcgaagatcatatcgcagaactgagtgtactgttgcagttggacgcggaggaagtgaggatggttggtttatggggttcctcagggatcggcaagactacaattgcaagagttctgtttcaacgactttctcgacacttccgaggtagcattttcatagacagggctttcgtatctaagactatggaaattttcaaggcagctaatccggacgactataacatgaagctgcatttgcaaagaaatttcctatctgaaatcttaggtaaaggagacataaagataaatcatttgagtgcagttggggagaggctgaagaatcagaaagttcttattttcattgatgattttgatgatcaagttgtgctagaagccttggttggtcaaactcaatggtttggaagtgggagcagaatcgttgtggttacaaatgataagcagtatctaagggcccatgggattaatcacatttacgaggtctgtctcccaactggaaacctagctgttgagatgttatgtcgatatgctttcaggaaaaaggctgcacctgaaggttttgaggagcttgtagctaaagttacaggacttgctggtagtcttcctttaggtcttaatgttttgggttcatctctacggggaagggacaaggagtactggatggatttgttgccaaggcttcggaattgtttagatgggaaaattgagaagacattgagagtcagctacgatggattaacaagcgaagaagataaagccttatttcgccatatcgcatgccttttcaatggtgcaacagtcacatacctgaagttggtgctcactgatagtgggttgagtgttaatgtggggctggaaaacctagctgataagtccctcattcatgaaagagaggattatgtggagatgcaccgtttgttagaagagatgggtagacgtattgttaggcttgaggagcctgaaaaacgagaatttctggtggacgcacaagatatctgtgatgtactcagtcaagacactgtaagttatctcttatgttcgtgctcctttcagtcaataaataagatttagagcatgccattttataagcaaaactaatacttgatattatataattttcagggtactcataagatattgggtataaaattgaatattgatgagattgatgaactgaatgtgcatgagaatgccttcaaagggatgcgcaatctgcgtttcctggaaattcactcaaaaaagcgttatgtgtttggaaaggaagaagtaccaattcacttacctgaaaacttcgactatttgcctccaaaacttaaaatattggattggtatgaatatccaatgagatgtctgccttctaagtttcgtcctgaaaaactcgtcaagctcaaaatggtgaatagcaagctcgagaagctgtggaaagggattgtggtaagttttgagaatagtttgtgatgttattagtagtaagactaatcttgattttattttttggatgacaatcttgttctactgagctcatgtgttttgttcctttttatttgttttgttgagtacagtcgcttacatgtcttaataagatggatatgtcggcatctcaaaacttgatagaaatgccagatctttcaaaggccaccaatctggagacacttaaacttcggaattgctatagtttggtcaagcttccttcctctattccacatcccaacaaactgacgacattaaacttgaagaactgtcgaaatctggagactattccaattggcattagcctcaaatctctcaaaaacctaaatactaaaggatgctcacggatgaggatttttccccaaatctcaaccagcatcgtagatgtcgacatagccgcaacatccattgaagaaataccttcaaatttgagtttgtgtttcgagagtctccatacctttacgatgcacagcccaaagaaactatgggaaagagtgcaggtatgtatgtagttccaaactttgtgtgtttctccaatctgttttacgttatagatattagataatggtgcaaatgaaactaaggttatattgtatttatcggagggaagaagagtagcgctgaatatgattttgtgtatttggttcagcttcttactctcctcacgacgatcatgtctccctctttgtggtatctggatctctcggataacccaggcttggtggagcttccttcttcatttaagaatctccataacttgcagatattgaaaattagtaactgcgtaaatctggaaactcttcccaccggaatcaacctcggatctctctggcaactagatctcagtggatgctcaaggttgaggacttttcctgatatctcaaccaacatcatagttctcgatctcagcgaaacagccatcgaagagattccttgttggattgagaaattctctaaccttaactcccttaggatgaagggatgcaacaatttggaatatgtaaacctaaacatttctaaactcaaacatcttcagaaagtagacttttcagactgcaagtcattgactggagctagcttgaataatcgtccaagagaaagtgccttgagttattaccacatatgctacattggtatcgatttcaccaagtgcttaaacttggatcaagaagctctgtttcaaaagaaaacatattttggttgtcatctgaagttgtcaggtgaagaagtgccttcatatttcactcaccgtactactggaacctcctcctctctcaccattcctttacttcacagctgtctctcacaaccattcctcctattcagggcttgtattgtgtttgattcggacaaggagacatatagcgattgtgtctttagattcaaaggcagttttcggaactgctctgattcctataatcaggcacaagacttctgcgcagtcacggatgattatgagatcaattcatatgagaaggatggttgtctgtttgtattagactaccagatgtctcaaatccctttagaaatgaacttcgatcgcctggatctgaagattcatattgttgattgttttaatgatgctaaaataaaaggatggggtatacgaatcttagaggaggactgttcatcggcagacaaccgacttggttatccaaacattctaccacatgtttttgaagccgatgaatgcaatatgaggctggtgaatgtggaggcaaatgatgcagtgacggaaagaagcgggtaaagggcattaatcatgaacttatcacagtattttctatatcataatttcttgtactgagactttttttttctttttctgcagatttcgtgaataaagttttgattgtgttaggttgtaacgcagagcatctaaaattaggaattaagagagtttcccatccaacttgcttgtggatcttagtatgcggagaagtaagagcacaatattgtgggaaggagtagtgcatgtatgttttcttcaaccttccttcctttatactagttcttacaaaaatggttatgcctggcagatctctagaaatgacctaactttattgtgtgtaccttaaattctcaggcagtgcctctttttctatcagagattgctcctgcacagctcaggggaggtctcaacattgtattccaactcatggtaacaattggaatcctaatagccaaccttgtcaactacttcactgccaccgttcaccctaacggatggcgaatcgccctcggtggagctgcaatccccgcggttatcctcctcttgggttcactgatcatctgtgagacccccacgagcctcatagagcgcaacaaaaacgaagaaggcagagaaactctaaggaaaatcagaggagttga

SEQ ID NO: 6 - protein, 1243 aa, Brassica napus
MALSLASSPSSSRTWLYDVFPSFSGVDVRVTFLSHLLKEFDKKLITAFKDNEIERSRSLDPELKQAIKDSRIAVVIFSQNYASSSWCLNELLEIVKRGQMVIPVFYRLDPSHVRKQTGDFGKIFEETCKNQKEEVIIIQWRRALTDVANTLGYHSVNWGNEAAMIEEIANDVLDKLLLTSSKDSENFVGIEDHIAELSVLLQLDAEEVRMVGLWGSSGIGKTTIARVLFQRLSRHFRGSIFIDRAFVSKTMEIFKAANPDDYNMKLHLQRNFLSEILGKGDIKINHLSAVGERLKNQKVLIFIDDFDDQVVLEALVGQTQWFGSGSRIVVVTNDKQYLRAHGINHIYEVCLPTGNLAVEMLCRYAFRKKAAPEGFEELVAKVTGLAGSLPLGLNVLGSSLRGRDKEYWMDLLPRLRNCLDGKIEKTLRVSYDGLTSEEDKALFRHIACLFNGATVTYLKLVLTDSGLSVNVGLENLADKSLIHEREDYVEMHRLLEEMGRRIVRLEEPEKREFLVDAQDICDVLSQDTGTHKILGIKLNIDEIDELNVHENAFKGMRNLRFLEIHSKKRYVFGKEEVPIHLPENFDYLPPKLKILDWYEYPMRCLPSKFRPEKLVKLKMVNSKLEKLWKGIVSLTCLNKMDMSASQNLIEMPDLSKATNLETLKLRNCYSLVKLPSSIPHPNKLTTLNLKNCRNLETIPIGISLKSLKNLNTKGCSRMRIFPQISTSIVDVDIAATSIEEIPSNLSLCFESLHTFTMHSPKKLWERVQLLTLLTTIMSPSLWYLDLSDNPGLVELPSSFKNLHNLQILKISNCVNLETLPTGINLGSLWQLDLSGCSRLRTFPDISTNIIVLDLSETAIEEIPCWIEKFSNLNSLRMKGCNNLEYVNLNISKLKHLQKVDFSDCKSLTGASLNNRPRESALSYYHICYIGIDFTKCLNLDQEALFQKKTYFGCHLKLSGEEVPSYFTHRTTGTSSSLTIPLLHSCLSQPFLLFRACIVFDSDKETYSDCVFRFKGSFRNCSDSYNQAQDFCAVTDDYEINSYEKDGCLFVLDYQMSQIPLEMNFDRLDLKIHIVDCFNDAKIKGWGIRILEEDCSSADNRLGYPNILPHVFEADECNMRLVNVEANDAVTERSGMRRSKSTILWEGVVHAVPLFLSEIAPAQLRGGLNIVFQLMVTIGILIANLVNYFTATVHPNGWRIALGGAAIPAVILLLGSLIICETPTSLIERNKNEEGRETLRKIRG

SEQ ID NO: 7 - DNA, 3585 bp, Brassica napus
  source 1..3585: mol_type = unassigned DNA
atggctctctcattagcttcttctccttcctcttctcgcacttggttgtacgatgttttccctagcttcagtggggtagacgttcgtgttactttcctcagccacttgttgaaggagtttgacaaaaagttgatcactgctttcaaagacaacgagatcgagagaagtcgatcactggatcccgagctcaaacaagccattaaagattcgaggatcgcagtggttatcttctcccaaaactatgcctcttcaagctggtgtcttaatgagttgttagagatagtcaagcgtggtcaaatggtgatacctgttttctaccggttggatccttcccacgtgaggaaacaaaccggtgaatttgggaaaatatttgaagaaacatgcaagaatcaaaaagaggaagtgataataactcaatggaggagagctttgaccgatgtagccaatactctcgggtatcattcggtaaactggggtaacgaagctgcaatgattgaagaaatcgccaatgatgttttggataaactacttttaacttcatcgaaggattcagagaactttgtgggcatcgaagatcatcttgcagaactgagtgtattgctgcagttggacgcggaggaagtgaggatggttggtttatggggttcctcagggatcggcaagactacaattgcaagagttctgtttcaacgactttctcgacacttccgaggtagcattttcatagacagggctttcgtatctaagactatggaaattttcaaggaagctaatccggacgactataacatgaagctgcatttgcaaagaaatttcctatctgaaatcttaggtaaaggagacataaagataaatcatttgagtgcagttgaagagaggctgaagaatcagaaagttcttattttcattgatgattttgatgatcaagttgtgctagaagccttggttggtcaaactcaatggtttggaagtgggagcagaatcgttgtggttacaaatgataagcagtatctaagggcccatgggattaatcacatttacgaggtctgtctcccaactgaaaacctagctgttgagatgttatgtcgatctgctttcaggaaaaaggctgcacctgaaggttttgaggagcttgtagctaaagttacaggacttgctggtagtcttcctttaggtcttaatgttttgggttcatatctacggggaagggagaagaagtactggatggatttgttgccaaggcttcagaatggtttagatgggaaaattgagaagacattgagggtcagctacgatggattaacaagcgaagaagataaagcgttatttcgccatatcgcatgccttttccaatggaaaaaagtcacatacctgaagttgctgctcgctgatagtgggttgagtgttacggttgggctggaaaacctagctgataagtccatcattcatgtaagtacgaattatgtggtgatgcaccgtttgttagaagagatgggtagaggtattgttaggcttgacgagcctgaaaaacgagaatttctggtggacgcacaagatatctgtgatgtactcagtcaagacactggtactcataagatattgggtataaaattgaatattgatgagattgatgaactgaatgtgcatgagaatgccttcaaagggatgcgcaatctgcgtttcctggaaattcactcacaaaaccgtcatgagtttggaaacgaagaagttagaattcacttacctgaaaacttcgactatttgcctccaaaacttaaaatattggattggtttggatatccaatgagatgtctgccttctaagtttcgtcctgaaaaactcgtcaagctcaaaatggtgaatagcaagctcgagaagctgtgggaagggattgtgtcgcttacatgtcttaaagagatggatatgtcgggatctacaaacttgatagaaatgccagatctttcaaaggccaccaatctggagacacttaatgttggggcttgctatagtttggtgacttttccccaaatctcaagcaccatcgtagatgtcgacatagccggaacatccattgaagaaataccttcaaatttgagtttgtgtttcgagaatctccatacctttaagatgcacagcccaaagaaactatcggaaagagtgcagcttcttactctcctcacgccgatcatgtctccctctttgtggtatctgaatctctcggataaccctggcttggtggagcttccttcttcatttaagaatctccataaactggagagattgaaaattagaaactgcgtaaatctggaaactcttcccaccggaatcaacctcggatctctctcgagagtagatctaaggggatgctcacggttgagaacttttcctgatatctcaaccaacatcaaaaacctcgatctcagcgaaacagccattgaagagattccttgttggattgagaaattctccaggctttactccctacggatgaagggatgcaacaatttggaatatgtaaacctaaacatttctaaactcaaatatcttttcgaagtcgacttttcagactgcaagtcattgactggagctagctggaataatcgtccaagagaaagtgccttgagttattaccacagtttcgacattggtatcgatttcaccaagtgcttaaacttggatcaagaagctctgtttcaaaagaaaacatatttcggttgtcaactgaagttgtcaggtgaagaagtgccttcatatttcacgcaccgtactactggaacctcctcctctctcaccattcctttacttcacagctgtctctcacaaccattcctccgattcagggcttgcattgtgtttgattcgcacaatgagacatatagcaaatgtgtctttagattcaaaggcagttttcagaactgctctgattcctataatcaggcacaagacttctgcgcagtcacggaggattatttgatctattcatatgagaaggatggttgtctgtttgtattagactaccagatgtctcaaatccctttagaaatgaacttcgatggcctggatctgaagattcatattgttgattgttataatgctaaaataaaaggatggggtatacgaatcttagaggaggactgttcatcggcagacaaccgacttggttatccaaacattctaccacatgtttttgaagccgatgaatgcaatatgaggctggtgaatgtggaggcaaatgatgcagtgacggaaagaagcggtatgcggagaagtaagagcacaatattgtgggaaggagtagtgcatgcagtgcctctttttctatcagagattgttcctgcacagctcaagggaggtctcaacattgtattccaactcatggtcacaattggaatcctaatagccaacctggtcaactacttcactgccaccgttcaccctaacggatcgcgaatcgccctcggtggagctgcaatccccgcggttatcctcctcttgggttcactgatcatctgtgagacccccacgagcctcatagagcgcaacaaaaacgaagaaggcagagaaactctaaggaagatcagagga

SEQ ID NO: 8 - DNA, 4344 bp, Brassica napus
  source 1..4344: mol_type = genomic DNA
atggctctctcattagcttcttctccttcctcttctcgcacttggttgtacgatgttttccctagcttcagtggggtagacgttcgtgttactttcctcagccacttgttgaaggagtttgacaaaaagttgatcactgctttcaaagacaacgagatcgagagaagtcgatcactggatcccgagctcaaacaagccattaaagattcgaggatcgcagtggttatcttctcccaaaactatgcctcttcaagctggtgtcttaatgagttgttagagatagtcaagcgtggtcaaatggtgatacctgttttctaccggttggatccttcccacgtgaggaaacaaaccggtgaatttgggaaaatatttgaagaaacatgcaagaatcaaaaagaggaagtgataataactcaatggaggagagctttgaccgatgtagccaatactctcgggtatcattcggtaaactggtacggttttatgcatcattcattcttttgagtaaaggttagacttctggttgcttgggggtgatattatctttcttattgttaggggtaacgaagctgcaatgattgaagaaatcgccaatgatgttttggataaactacttttaacttcatcgaaggattcagagaactttgtgggcatcgaagatcatcttgcagaactgagtgtattgctgcagttggacgcggaggaagtgaggatggttggtttatggggttcctcagggatcggcaagactacaattgcaagagttctgtttcaacgactttctcgacacttccgaggtagcattttcatagacagggctttcgtatctaagactatggaaattttcaaggaagctaatccggacgactataacatgaagctgcatttgcaaagaaatttcctatctgaaatcttaggtaaaggagacataaagataaatcatttgagtgcagttgaagagaggctgaagaatcagaaagttcttattttcattgatgattttgatgatcaagttgtgctagaagccttggttggtcaaactcaatggtttggaagtgggagcagaatcgttgtggttacaaatgataagcagtatctaagggcccatgggattaatcacatttacgaggtctgtctcccaactgaaaacctagctgttgagatgttatgtcgatctgctttcaggaaaaaggctgcacctgaaggttttgaggagcttgtagctaaagttacaggacttgctggtagtcttcctttaggtcttaatgttttgggttcatatctacggggaagggagaagaagtactggatggatttgttgccaaggcttcagaatggtttagatgggaaaattgagaagacattgagggtcagctacgatggattaacaagcgaagaagataaagcgttatttcgccatatcgcatgccttttccaatggaaaaaagtcacatacctgaagttgctgctcgctgatagtgggttgagtgttacggttgggctggaaaacctagctgataagtccatcattcatgtaagtacgaattatgtggtgatgcaccgtttgttagaagagatgggtagaggtattgttaggcttgacgagcctgaaaaacgagaatttctggtggacgcacaagatatctgtgatgtactcagtcaagacactgtaagttatctcttatgttcgtgctccttacagtcaataaataagatttagagcatgccattttataagcaaaactaatacttgatattatataattttcagggtactcataagatattgggtataaaattgaatattgatgagattgatgaactgaatgtgcatgagaatgccttcaaagggatgcgcaatctgcgtttcctggaaattcactcacaaaaccgtcatgagtttggaaacgaagaagttagaattcacttacctgaaaacttcgactatttgcctccaaaacttaaaatattggattggtttggatatccaatgagatgtctgccttctaagtttcgtcctgaaaaactcgtcaagctcaaaatggtgaatagcaagctcgagaagctgtgggaagggattgtggtaagttttgagaatagtttgtgatgttatttgtagtaagactaatctttattttattttttggatgacaatcttgttctactgagctcatgtgttctgttcctttttttttttgttgagtacagtcgcttacatgtcttaaagagatggatatgtcgggatctacaaacttgatagaaatgccagatctttcaaaggccaccaatctggagacacttaatgttggggcttgctatagtttggtgacttttccccaaatctcaagcaccatcgtagatgtcgacatagccggaacatccattgaagaaataccttcaaatttgagtttgtgtttcgagaatctccatacctttaagatgcacagcccaaagaaactatcggaaagagtgcaggtatgtatgtagttccaaactttgtgtgtttctccaatctgttttacgttatagatattagatattggtgaaaatgaaactaaggttatattgtatttatcggagggaagaagagtagcgctgaatatgattttgtgtatttggttcagcttcttactctcctcacgccgatcatgtctccctctttgtggtatctgaatctctcggataaccctggcttggtggagcttccttcttcatttaagaatctccataaactggagagattgaaaattagaaactgcgtaaatctggaaactcttcccaccggaatcaacctcggatctctctcgagagtagatctaaggggatgctcacggttgagaacttttcctgatatctcaaccaacatcaaaaacctcgatctcagcgaaacagccattgaagagattccttgttggattgagaaattctccaggctttactccctacggatgaagggatgcaacaatttggaatatgtaaacctaaacatttctaaactcaaatatcttttcgaagtcgacttttcagactgcaagtcattgactggagctagctggaataatcgtccaagagaaagtgccttgagttattaccacagtttcgacattggtatcgatttcaccaagtgcttaaacttggatcaagaagctctgtttcaaaagaaaacatatttcggttgtcaactgaagttgtcaggtgaagaagtgccttcatatttcacgcaccgtactactggaacctcctcctctctcaccattcctttacttcacagctgtctctcacaaccattcctccgattcagggcttgcattgtgtttgattcgcacaatgagacatatagcaaatgtgtctttagattcaaaggcagttttcagaactgctctgattcctataatcaggcacaagacttctgcgcagtcacggaggattatttgatctattcatatgagaaggatggttgtctgtttgtattagactaccagatgtctcaaatccctttagaaatgaacttcgatggcctggatctgaagattcatattgttgattgttataatgctaaaataaaaggatggggtatacgaatcttagaggaggactgttcatcggcagacaaccgacttggttatccaaacattctaccacatgtttttgaagccgatgaatgcaatatgaggctggtgaatgtggaggcaaatgatgcagtgacggaaagaagcgggtaaaggcattaatcatgaacttatcacagtattttctatatcataatttcttgtactgagacttttttttctttttctgcagatttcgtgaataaagttttgattgtgttaggttgtaacgcagagcatctaaaattaggaattaagagagtttcccatccaacttgcttgtggatcttagtatgcggagaagtaagagcacaatattgtgggaaggagtagtgcatgtatgttttcttcaaccttccttccttttatactagttcttacaaaaatggttatgcctggcagatctctagaaatgaccaaacattattgtgtgtaccttaaattctcaggcagtgcctctttttctatcagagattgttcctgcacagctcaagggaggtctcaacattgtattccaactcatggtcacaattggaatcctaatagccaacctggtcaactacttcactgccaccgttcaccctaacggatcgcgaatcgccctcggtggagctgcaatccccgcggttatcctcctcttgggttcactgatcatctgtgagacccccacgagcctcatagagcgcaacaaaaacgaagaaggcagagaaactctaaggaagatcagaggagttga

SEQ ID NO: 9 - protein, 1195 aa, Brassica napus
MALSLASSPSSSRTWLYDVFPSFSGVDVRVTFLSHLLKEFDKKLITAFKDNEIERSRSLDPELKQAIKDSRIAVVIFSQNYASSSWCLNELLEIVKRGQMVIPVFYRLDPSHVRKQTGEFGKIFEETCKNQKEEVIITQWRRALTDVANTLGYHSVNWGNEAAMIEEIANDVLDKLLLTSSKDSENFVGIEDHLAELSVLLQLDAEEVRMVGLWGSSGIGKTTIARVLFQRLSRHFRGSIFIDRAFVSKTMEIFKEANPDDYNMKLHLQRNFLSEILGKGDIKINHLSAVEERLKNQKVLIFIDDFDDQVVLEALVGQTQWFGSGSRIVVVTNDKQYLRAHGINHIYEVCLPTENLAVEMLCRSAFRKKAAPEGFEELVAKVTGLAGSLPLGLNVLGSYLRGREKKYWMDLLPRLQNGLDGKIEKTLRVSYDGLTSEEDKALFRHIACLFQWKKVTYLKLLLADSGLSVTVGLENLADKSIIHVSTNYVVMHRLLEEMGRGIVRLDEPEKREFLVDAQDICDVLSQDTGTHKILGIKLNIDEIDELNVHENAFKGMRNLRFLEIHSQNRHEFGNEEVRIHLPENFDYLPPKLKILDWFGYPMRCLPSKFRPEKLVKLKMVNSKLEKLWEGIVSLTCLKEMDMSGSTNLIEMPDLSKATNLETLNVGACYSLVTFPQISSTIVDVDIAGTSIEEIPSNLSLCFENLHTFKMHSPKKLSERVQLLTLLTPIMSPSLWYLNLSDNPGLVELPSSFKNLHKLERLKIRNCVNLETLPTGINLGSLSRVDLRGCSRLRTFPDISTNIKNLDLSETAIEEIPCWIEKFSRLYSLRMKGCNNLEYVNLNISKLKYLFEVDFSDCKSLTGASWNNRPRESALSYYHSFDIGIDFTKCLNLDQEALFQKKTYFGCQLKLSGEEVPSYFTHRTTGTSSSLTIPLLHSCLSQPFLRFRACIVFDSHNETYSKCVFRFKGSFQNCSDSYNQAQDFCAVTEDYLIYSYEKDGCLFVLDYQMSQIPLEMNFDGLDLKIHIVDCYNAKIKGWGIRILEEDCSSADNRLGYPNILPHVFEADECNMRLVNVEANDAVTERSGMRRSKSTILWEGVVHAVPLFLSEIVPAQLKGGLNIVFQLMVTIGILIANLVNYFTATVHPNGSRIALGGAAIPAVILLLGSLIICETPTSLIERNKNEEGRETLRKIRG

SEQ ID NO: 10 - DNA, 8102 bp, synthetic construct
  source 1..8102: mol_type = other DNA
  misc_feature 1..8102: note = nucleotide sequence of the T-DNA 35S::CRT
aattacaacggtatatatcctgccagtactttgtggcgctctatcatagctataaacctattcagcacaatatcgattaagggccccctcgagggcgatcgctacgtacctgcaggcccgggttaattaagcggccgcaacatggagtcaaaaattcagatcgaggatctaacagaactcgccgtgaagactggcgaacagttcatacagagtcttttacgactcaatgacaagaagaaaatcttcgtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcccctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggactcgagctcatttctctattacttcagccataacaaaagaactcttttctcttcttattaaaccaaaaccatggctctctcattagcttcttctccttcctcttgtcgcacttggttgtacgatgttttccctagcttcagtggggtagacgttcgtgttactttcctcagccacttgttgaaggagtttgacaaaaagttgatcactgctttcaaagacaacgagatcgagagaagtcgatcactggatcccgagcttaaacaagccattaaagattcgaggatcgcagtggttatcttctcccaaaactatgcctcttcaagctggtgtcttaatgagttgttagagatagtcaagtgtggtcaaatggtgatacctgttttctaccggttggatccttcccatgtgaggaaacaaaccggtgactttggtaagttctttgaagaaacatgcaacaacaaaacagaggaagagaaaatacagtggaggagagctttgaccgatgtagccaatactctcgggtatcattcagtaaactggtacggttttatgcttcttttgaataaaggttagacttctggttgctagggggtgatattatctttcttattgttaggggtaacgaagctgcaatgattgaagaaatcgccaatgatgttttggataaactacttttaacttcatcgaaggattcagagaactttgtgggcatcgaagatcatgttgcaaaactgagtgtattgctgcagttggacgcggaggaagtgaggatggttggtttatggggttcctcagggatcggcaagactacaattgcaagagttctgtttcaacgactttctcgacacttccgaggtagcattttcatagacagggctttcgtatctaagactatggaaattttcaaggcagctaatccggacgactataacatgaagctgcatttgcaaagaaatttcctatctgaaatcttaggtaaaggagacataaagataaatcatttgagtgcagttggggagaggctgaagaatcagaaagttcttattttcattgatgattttgatgatcaagttgtgctagaagccttggttggtcaaactcaatggtttggaagtgggagcagaatcgttgtggttacaaatgataagcagtatctaagggcccatgggattaatcacatttacaaggtctgtctcccaactaaaaagctagctgttgagatgttatgtcgatctgctttcaggaaaaaggctgcacctgaaggttttgaggagcttgtagctaaagttacaggacttgctggtagtcttcctttaggtcttaatgttttgggttcatatctacggggaagggacaaggagtactggatggatttgttgccaaggcttcagaatggtttagatgggaaaattgagaagacattgagagtcagctacgatggattaacaagcgaagaagataaagcgttatttcgccatattgcatgccttttccagtgggaaaaagtcacatacctgaagttgctgctcgctgatagtgggttgagtgttacggttgggctggaaaacctagctgataagtccctcattcatgtaagagaggattatgtgaagatgcaccgtttgttagaagagatgggtagacgtattgttaggcttgacgagcctgaaaaacgagaatttctggtggacgcacaagatatctgtgatgtactcagtcaagacactgtaagttatctcttatgttcgtgctcctttcagtcaataaataagatttagagcatgccattttataagcaaaactaatacttgatattatataattttcagggtactcataagatattgggtataaaattgaatattgatgagattgatgaactgaatgtgcatgagaatgccttcaaagggatgcgcaatctgcgtttcctggaaattcactcacaaaaccgtcatgagtttggaaacgaagaagttagaattcacttacctgaaaacttcgactatttgcctcctaaacttaaaatattggattggtatgaatatccaatgagatgtctgccttctaagtttcgtcctgaaaaactcgtcaagctcaaaatggtgaatagcaagctcgagaagctgtgggaagggattgtggtaagttttgagaatagtttgtgatgttatttgtagtaagactaatctttattttattttttggatgacaatcttgttctactgagctcatgtgttctgttcctttttttttttgttgagtacagtcgcttacatgtcttaaaaagatgaatatgtcgggatctcaaaacttgatagaaatgccagatctttcaaaggccaccaatctggagacactatatcttgaggattgctttagtttggtcaagcttccttcctctattccacaccccaacaaactgacgacattaatcttgaagaactgtcgaaatgtggagactattccaattggcattagcctcaaatctcttaaaaacctacgtactgatggttgctcacggatgaggacttttccccaaatctcaagcaccatcgaagatgtctacataggcgcaacatccattgaagaaataccttcaaatttgagtttgtgtttcgagaatctccatacctttacgatgcacagcccaaagaaactatgggaaagagtgcaggtatgtatgtagttccaaactttgtgtgtttctccaatctgttttacgttatagatattagataatggtgcaaatgaaactaaggttatattgtatttatcggagggaagaagagtagcgctgaatatgattttgtgtatttggttcagcttcttactctcctcacgacgatcatgtctccctctttgtggtatctggatctctcggataaccctggcttggtggagcttccttcttcatttaagaatctccataacctgaggagattggaaattagaaactgcgtaaatctggaaactcttcccaccggaatcaacctcggatctctcaaaatcctagatctcaggggatgctcacggttgaggacttttcctgatatctcaacccacatcacacatctttatctaagcggaacagggattgaagagattccttgttcgattgagaaattctccaggcttggctccctacatatgaacggatgcaacaatttggaatatgtaaacctaaacctttttaaactcaaacatcttcacgaagtcgacttttcagactgcaagtgcttaaacttggatcaagaagctctgtttcaaaagaaaacatattcagtttgtcaactgaagttgtcaggtgaagaagtgccttcatatttcacgcaccgtactactggaacctcctcctctctcaccattcctctacttcacagctgtatctcacaatcattcctccgattcagggcttgtattgtgtttgattcggacaaggacaatgagtcatatagcagatgtgcctttagattcaaaggcagttttcggaactgctctgattcctataatcaggcacaagacttctgcgcagtcacggatgattataagatccgttcatataagaaggatggttgtctgcttgtattagactaccagatgtctcaaatccctttagaaatgaacttcgatggcctggatctgaagattcatattgattattgtcgttctgctaaaataaaaggatggggtatacgaatcttagaggaggactgttcatcggcagacaaccgacttggttatccaaacattctaccacatgtttttgaagccgatgaatgcaatgaggctggtgaatgtgggaggcaaatgatgtagtgacggaaagaagcgggtaaaggcattaatcatgaacttatcacagtattttctatatcataatttcttgtactgagacttttttttctttttctgcagatttcgtgaataaacttttgattgtgttaggttgtaacgcagagcatctaaaattaggaattaagagagattcccatccaacttgcttgtggatcttagtatgcggagaagtaagagcacaaaattgtgggaaggagtagtgcatgtatgttttcttcaaccttccttcctttatactagttcttacaaaaatggttatgcctggcagatctctagaaatgacctaacattattgtgtgtaccttaaattctcaggcagtgcctctttttctatcagagattgctcctgcacagctcaggggaggtctcaacattgtattccaactcatggtcacaattggaatcctaatagccaaccttgtcaactacttcactgccaccgttcaccctaacggatggcgaatcgccctcggtggagccgcaatccccaccgttatcctagtcttcggttcactgatcatctgcgagactcccacgagcttcatagagcgcaagtgttgaaaacgcgtggcgcgcctaagctagctatatcatcaatttatgtattacacataatatcgcactcagtctttcatctacggcaatgtaccagctgatataatcagttattgaaatatttctgaatttaaacttgcatcaataaatttatgtttttgcttggactataatacctgacttgttattttatcaataaatatttaaactatatttctttcaagatgaattcgatatcattaccctgttatccctaaagcttattaatataacttcgtatagcatacattatacgaagttatgtttcaaatttattatgtgttttttttccgtggtcgagattgtgtattattctttagttattacaagacttttagctaaaatttgaaagaatttactttaagaaaatcttaacatctgagataatttcagcaatagattatatttttcattactctagcagtatttttgcagatcaatcgcaacatatatggttgttagaaaaaatgcactatatatatatatattattttttcaattaaaagtgcatgatatataatatatatatatatatatatatgtgtgtgtgtatatggtcaaagaaattcttatacaaatatacacgaacacatatatttgacaaaatcaaagtattacactaaacaatgagttggtgcatggccaaaacaaatatgtagattaaaaattccagcctccaaaaaaaaatccaagtgttgtaaagcattatatatatatagtagatcccaaatttttgtacaattccacactgatcgaatttttaaagttgaatatctgacgtaggatttttttaatgtcttacctgaccatttactaataacattcatacgttttcatttgaaatatcctctataattatattgaatttggcacataataagaaacctaattggtgatttattttactagtaaatttctggtgatgggctttctactagaaagctctcggaaaatcttggaccaaatccatattccatgacttcgattgttaaccctattagttttcacaaacatactatcaatatcattgcaacggaaaaggtacaagtaaaacattcaatccgatagggaagtgatgtaggaggttgggaagacaggcccagaaagagatttatctgacttgttttgtgtatagttttcaatgttcataaaggaagatggagacttgagaagttttttttggactttgtttagctttgttgggcgttttttttttttgatcaataactttgttgggcttatgatttgtaatattttcgtggactctttagtttatttagacgtgctaactttgttgggcttatgacttgttgtaacatattgtaacagatgacttgatgtgcgactaatctttacacattaaacatagttctgttttttgaaagttcttattttcatttttatttgaatgttatatatttttctatatttataattctagtaaaaggcaaattttgcttttaaatgaaaaaaatatatattccacagtttcacctaatcttatgcatttagcagtacaaattcaaaaatttcccatttttattcatgaatcataccattatatattaactaaatccaaggtaaaaaaaaggtatgaaagctctatagtaagtaaaatataaattccccataaggaaagggccaagtccaccaggcaagtaaaatgagcaagcaccactccaccatcacacaatttcactcatagataacgataagattcatggaattatcttccacgtggcattattccagcggttcaagccgataagggtctcaacacctctccttaggcctttgtggccgttaccaagtaaaattaacctcacacatatccacactcaaaatccaacggtgtagatcctagtccacttgaatctcatgtatcctagaccctccgatcactccaaagcttgttctcattgttgttatcattatatatagatgaccaaagcactagaccaaacctcagtcacacaaagagtaaagaagaacaatggacccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctgatctcacccgtctaggatcccccgatgagctaagctagctatatcatcaatttatgtattacacataatatcgcactcagtctttcatctacggcaatgtaccagctgatataatcagttattgaaatatttctgaatttaaacttgcatcaataaatttatgtttttgcttggactataatacctgacttgttattttatcaataaatatttaaactatatttctttcaagataaacataacttcgtatagcatacattatacgaagttatcaaaacgtcgtgagacagtttggttaactataacggtcctaaggtagcgatcgaggcattacggcattacggcactcgcgagggtccgaatctatgtcgggtgcggagaaagaggtaatgaaatggcaattcgagcatggagccatttacaattgaatatatcctgccg

SEQ ID NO: 11 - DNA, 121 bp, Brassica napus
  source 1..121: mol_type = genomic DNA
atgatctttcatcaaaatatcggatgagaaagcttgaagcttcgggactgctcatatactattaagkaaaatagtatttgtgtataaagaagtctattagttagtaaacaaattgagagaa

SEQ ID NO: 12 - DNA, 121 bp, Brassica napus
  source 1..121: mol_type = genomic DNA
cttctggtttgatacckgttmagmaaatgtcatcggtcrgtctgatttgattgaagaaacgggtaagttgtgcagagactgtgaagctcaaggcataggcatagctagcaattattcaacc

(19) United States
(12) Patent Application Publication
(10) Pub. No.: US 2025/0084425 A1
(43) Pub. Date: Mar. 13, 2025

(54) BRASSICA PLANT RESISTANT TO PLASMODIOPHORA BRASSICAE (CLUBROOT)

(71) Applicant: BASF Agricultural Solutions Seed US LLC, Florham Park, NJ (US)

(72) Inventors: Steven ENGELEN, Gent (BE); Stephen Rae, Gent (BE); Godfrey Chongo, Saskatoon (CA); Jasper Devlamynck, Deinze (BE); Kim Crommar, Gent (BE); Katrien Van Audenhove, Gent (BE)

(21) Appl. No.: 18/958,890

(22) Filed: Nov. 25, 2024

Related U.S. Application Data
(62) Division of application No. 16/981,196, filed on Sep. 15, 2020, now Pat. No. 12,163,136.

(30) Foreign Application Priority Data
Mar. 16, 2018 (EP) 18162162.4

(51) Int. Cl. C12N 15/82; A01H 1/00; A01H 5/10; A01H 6/20; C12Q 1/68

(52) U.S. Cl. CPC C12N 15/82; A01H 1/125; A01H 5/10; C12Q 1/68; A01H 6/20

(57) ABSTRACT

The present invention relates to clubroot resistance in Brassica. More specifically, the invention relates to the CRT clubroot resistance gene, as well as to methods to create clubroot resistant Brassica plants.

### CROSS-REFERENCE TO RELATED APPLICATIONS

This application is a divisional of U.S. patent application Ser. No. 16/981,196, filed on Sep. 15, 2020, which is a National Stage Entry of PCT/US2019/022601, filed Mar. 15, 2019, which claims priority to EP application Ser. No. 18/162,162.4, filed Mar. 16, 2018, the disclosures of each of which are hereby incorporated by reference in their entireties.

### REFERENCE TO SEQUENCE LISTING SUBMITTED ELECTRONICALLY

The sequence listing of the present application is submitted electronically as an XML file named “039621-01318_SequenceListing.xml”, created on Oct. 30, 2024, and having a size of 48,317 bytes. This sequence listing submitted electronically is an integral part of the specification and is incorporated herein by reference in its entirety.

### FIELD OF THE INVENTION

The invention relates to the field of disease control in Brassicacea. Provided are methods for the production of clubroot resistant plants through introduction of a clubroot resistance gene in their genome. Also provided are B. napus plants and seeds comprising one or more clubroot resistance loci in their genome. Further provided are detection tools for detecting the presence of one or more resistance alleles in B. napus plants, tissue or seeds, as well as methods for transferring one or more resistance loci to other Brassica plants and methods for combining different resistance loci in hybrid seeds and plants. Methods for enhancing durability of resistance to Plasmodiophora brassicae are also provided, as well as uses of the plants and seeds and the processes or kits of the invention.

### BACKGROUND OF THE INVENTION

Clubroot is a disease caused by Plasmodiophora brassicae which affects the Brassicaceae family of plants, including many important vegetable and broad acre crops. All members of the family Brassicaceae are thought to be potential hosts for Plasmodiophora brassicae (Dixon, 2009, J Plant Growth Regul 28:194). Susceptible cultivated crops include all varieties of B. oleracea, the Occidental Cole vegetables (Brussels sprout, cabbages, calabrese/green broccoli, cauliflower, culinary and fodder kale, kohlrabi); B. rapa (syn. B. campestris) including turnip, turnip rape, sarson, and the enormous range of Oriental variants which provide leaf and root vegetables such as Brassica rapa var. pekinensis and B. rapa var. chinensis (Chinese cabbages); B. napus including swede (rutabaga), oil seed rape, and fodder rape; and seed, condiment (mustard), and vegetable crops derived from B. carinata, B. nigra, and B. juncea. Related genera such as radish (Raphanus), cruciferous weeds, for example, Sinapis, and decorative ornamentals including stocks (Matthiola spp) and wallflower (Cheiranthus cheiri) can be infected. The scientific model plant Arabidopsis is also susceptible (Dixon, 2009, supra).

Clubroot disease symptom development is characterized by the formation of club-shaped galls on the roots of affected plants. As a result, the nutrient and water uptake by infected roots is inhibited. Above-ground symptoms include wilting, stunting, yellowing and premature senescence (Hwang et al, 2012, Mol Plant Pathol 13:105).

Clubroot disease is estimated to be present in approximately 10% of all areas where host plants are cultivated (Diederichsen et al, 2009, J Plant Growth Regul 28:265). Clubroot has been largely a disease of vegetable crops in the last century. However, in 2003, 12 clubroot-infested commercial fields were found in the central part of the province of Alberta. Thereafter, the number of fields with confirmed clubroot infestations has increased steadily, and, by 2010, more than 560 fields (over 35 000 ha) in Alberta had been identified as being infested with P. brassicae (Hwang et al., 2012, supra). Yield losses of 80%-91% were reported in studies with canola grown on clubroot-infested fields in Quebec. Seed quality was also reduced significantly, with declines of 4.7%-6.1% in oil content and 13%-26% in 1000-seed weights (Hwang et al., 2012, supra).

Plant resistance is a powerful tool to combat clubroot disease. Breeding for clubroot resistance focuses today on Chinese cabbage (B. rapa spp. Pekinensis) in Japan and Korea, oilseed rape in Germany and Sweden, and several B. oleracea vegetables. Recently released resistant cultivars belong to three Brassica species: B. napus, B. oleracea, and B. rapa (Diederichsen et al., 2009, supra).

Resistant sources of the European fodder turnips (B. rapa ssp, rapifera) have been identified, which have been used to transfer the clubroot resistance genes to Chinese cabbage. At least three independent dominant genes, which confer differential (race-specific or vertical) resistance to particular pathotypes of P. brassicae, appear to be present in turnip genotypes (Piao et al., 2009, J Plant Growth Regul 28:252). Eight possible clubroot resistance genes present in B. rapa have been identified through QTL mapping: CRa from resistant source ECD02, CRb from Gelria R. Crr1, Crr2 and Crr4 from Siloga, Crr3 from Milan White, and CRk and CRc from Debra. Crr1, Crr2, Crr3, Crr4 and CRc are mapped to chromosomes R8, R1, R3, R6 and

R2, respectively. CRa, CRb and CRk with Crr3 are mapped on the same linkage group of R3, but they are not located in the same chromosome region, except for CRk and Crr3 (Piao et al., 2009, supra; Sakamoto et al., 2008, Theor Appl Genet 117:759).

In B. oleracea, completely resistant accessions have been rarely identified. The inheritance of the clubroot resistance in B. oleracea appears polygenic and controlled by many dominant alleles with predominance of additive effects of with incomplete dominance. It has also been suggested that one of the resistances studied is controlled by two complementary genes (Piao et al., 2009, supra). At least 22 QTLs have been found in B. oleracea, indicating a complex genetic basis of clubroot resistance in B. oleracea. As the different mapping studies used different clubroot resistance sources and different P. brassicae isolates, a comparison of these QTLs is not possible (Piao et al., 2009, supra).

Clubroot resistance has also been observed in several B. napus cultivars. At least 22QTLs for clubroot resistance have been identified in B. napus. A major gene, Pb-Bn1, has been mapped onto linkage group DY4, and at least two additive QTLs have been identified on chromosomes DY4 and DY15, respectively. In addition, epistatic interactions between nine regions with or without additive effects have been located. A major gene and two recessive genes derived from ECD04 have been identified in double-haploid populations. In resynthesized B. napus developed by crossing cv. Böhmerwaldkohl (B. oleracea) and ECD-04 (B. rapa), nineteen QTLs expressing resistance to seven isolates were detected on eight chromosomes, four of which were closely linked to each other on chromosome NO3, and three were linked on chromosome N08. Genes CRk and Crr3 are located in the similar region of PbBn-k-2, PbBn-1-1,and PbBn-01:60-1 on NO3. CRa and CRb are independent from them. PbBn-01.07-2, PbBn-1-2,and PbBn-a-1 are linked to BRMS088 on chromosome N08 in B. napus, which is also linked with Crrl on R8 in B. rapa. PbBn-k-1 is located on chromosome NO2. The QTLs located on N03 and N19 contribute strong effects and confer broad-spectrum resistance (Piao et al., 2009,supra; and Werner et al., 2008, Theor Appl Genet 116:363). A clubroot resistant loci has also been recently identified which relates to resistance to various Plasmodiophora brassicae pathotypes including pathotype 5x (WO2016/176358).

The CRa gene of Brassica rapa has been fine-mapped and a TIR-NBS-LRR gene has been identified as the CRa gene (Ueno et al., 2012, Plant Mol Biol 80:621). The Crr1 gene has been mapped and isolated from the B. rapa European fodder turnip “Siloga”. Crr1a also encodes a TIR-NB-LRR disease resistance protein (Hatakeyama et al., 2013, PLOS one 8:e54745 and WO2012/039445).

The CRb gene from B. rapa has been fine-mapped to a 140 kb genomic region. In this region, in which fourteen functional proteins were predicted, amongst which are a Rho family proteins and two TIR-NBS-LRR proteins, which could be candidate genes for CRb (Kato et al., 2013, Breeding Science 63:116).

To increase the durability of clubroot-resistant cultivars, the combination of the different clubroot resistance genes into a single line will be an important means for breeding cultivars with resistance to a broader spectrum of physiological races. Therefore, in order to stack genes without linkage drag using marker-assisted selection and transgenic approaches, there remains a need to develop molecular markers linked to the clubroot resistance genes. This invention provides the sequence of a clubroot resistance locus from a resistant Brassica napus line, as herein after described in the different embodiments, examples and claims.

### SUMMARY OF THE INVENTION

In one aspect, the invention provides a protein capable of conferring clubroot resistance comprising an amino acid sequence selected from the group consisting of a) an amino acid sequence of SEQ ID NO: 3 and b) an amino acid sequence having at least 95% sequence identity with the amino acid sequence of SEQ ID NO: 3. An isolated nucleic acid molecule comprising a nucleotide sequence which encodes the protein of the invention is furthermore provided, that is selected from the group consisting of a) a nucleic acid comprising a nucleotide sequence of SEQ ID NO: 1 or SEQ ID 2, b) a nucleic acid sequence having at least 80% identity to the nucleic acid sequence of SEQ ID NO: 1 or SEQ ID NO: 2 and c) a nucleic acid having a complementary sequence to the nucleic acid of a) or b).

It is another object of the present invention to provide a recombinant gene comprising a plant expressible promoter operably linked to a nucleic acid sequence encoding the protein of the invention and optionally, a transcription termination and polyadenylation sequence, preferably a transcription termination and polyadenylation region functional in plants. In another object, said nucleic acid is selected from the group consisting of a) a nucleic acid comprising a nucleotide sequence of SEQ ID NO: 1 or SEQ ID NO: 2; b) a nucleic acid sequence having at least 80% identity to the nucleic acid sequence of SEQ ID NO: 1 or SEQ ID NO: 2; and c) a nucleic acid having a complementary sequence to the nucleic acid of a) or b). In another embodiment, said plant expressible promoter is selected from the group consisting of constitutive promoter, inducible promoter, tissue specific promoter. The plant expressible promoter may be the CaMV35S promoter.

The present invention furthermore provides a plant or plant cells and seeds comprising the recombinant gene according to the invention. The described plant may be a Brassicacae, which may be selected from the group consisting of Brassica napus, Brassica juncea, Brassica oleracea, Brassica rapa, Brassica nigra and Brassica carinata, and may further comprise at least one other disease resistance gene selected from the group consisting of a clubroot resistance gene, a blackleg resistance gene, a Sclerotinia resistance gene, a

Verticillium stripe resistance gene, a Fusarium wilt resistance gene, an Aster Yellows resistance gene, an Alternaria resistance gene, and a Grey Stem resistance gene.

The invention further provides a method for obtaining a clubroot resistant Brassicacae plant, comprising a) introducing or providing the clubroot resistance gene encoding the protein according to the invention to a Brassicacae plant cell, to create a Brassicacae cell, and b) regenerating a plant from said cell. Said Brassicacae plant may be selected from the group consisting of Brassica napus, Brassica juncea, Brassica oleracea, Brassica rapa, Brassica nigra and Brassica carinata. In another embodiment, the clubroot resistance gene is introduced or provided to the Brassicacae plant cell by providing or introducing to the Brassicacae plant cell the recombinant gene according to the invention. In yet another embodiment, the invention provides another method for obtaining a clubroot resistant Brassicacae plant, comprising a) providing a first Brassica plant that comprises the clubroot resistance sequence according to the invention; b) providing a second Brassica plant that lacks the clubroot resistance sequence of the invention; c) crossing the first Brassica plant with the second Brassica plant to provide progeny Brassica plant; and d) selecting Brassica progeny plant that tests positive for the presence of the clubroot resistance sequence according to the invention as being Brassica plant into which the clubroot resistance sequence of the invention has been introduced.

It is another object of the invention to provide a Brassicacae plant obtained by the methods according to the invention. In a further aspect, the Brassicacae plant comprises the clubroot resistance sequence according to the invention and at least one other disease resistance gene, said other disease resistance gene selected from the group consisting of a clubroot resistance gene, a blackleg resistance gene, a Sclerotinia resistance gene, a Verticillium resistance gene, a Fusarium wilt resistance gene, an Aster Yellows resistance gene, an Alternaria resistance gene, and a Grey Stem resistance gene. Such Brassicacae plant may be a Brassica napus. Seeds of this Brassicacae plant are also provided and they may be hybrid seeds. With such hybrid seeds, being Brassica napus hybrid seeds, and developing into plants, the solid component of the seeds may contain less than 30 micromoles of any one or any mixture of 3-butenyl glucosinolate, 4-pentenyl glucosinolate, 3-hydroxy-3 butenyl glucosinolate, and 2-hydroxy-4-pentenyl glucosinolate per gram of air-dry, oil-free solid.

Further embodiments disclose a kit for the detection of a CRT clubroot resistance locus in Brassica DNA samples, wherein said kit comprises one or more PCR primer pairs, which are able to amplify a DNA marker linked to CRT. The disclosed kit may comprise two primers recognizing CRT and not recognizing the nucleotide sequence of SEQ ID NOs: 4, 5, 7or 8.

Yet another embodiment provides a method of producing food, feed, or an industrial

product comprising obtaining the plant according to the invention or a part thereof, and preparing the food, feed or industrial product from this plant or part thereof. In a further object, said food or feed is oil, meal, grain, starch, flour or protein; or said industrial product is biofuel, fiber, industrial chemicals, a pharmaceutical or a nutraceutical.

### BRIEF DESCRIPTION OF THE FIGURES

FIG. 1. Alignment of the nucleotide sequence of the CRT gene (SEQ ID NO: 2) with the CRS genes (SEQ ID NOs: 5 and 8). Nucleotides conserved between CRT and at least one CRS are indicated by an asterisk.

FIG. 2. Alignment of the amino acid sequence of the CRT protein (SEQ ID NO: 3) with the CRS proteins (SEQ ID NOs: 6 and 9). Amino acid residues conserved in all proteins are indicated by an asterisk, conserved amino acid substitutions between CRT and at least one CRS are indicated by a semi colon, amino acids present in one of the CRS but absent from both CRT and the other CRS are indicated with a dash. The TIR domain in SEQ ID NO: 3 is indicated by a grey shadow, the NB-ARC domain is indicated by a box, and the LRR domain is indicated by an underline.

FIG. 3. Result of the clubroot resistance assay performed on three transgenic lines (events) expressing CRT (A) and their respective null segregant (B) at 60 dpi (days post infection).

### DETAILED DESCRIPTION

The current invention is based on the identification of a CRT clubroot resistance gene in Brassica.

In one aspect, the invention provides a protein capable of conferring clubroot resistance comprising an amino acid sequence selected from the group consisting of a) an amino acid sequence of SEQ ID NO: 3 and b) an amino acid sequence having at least 95% sequence identity with the amino acid sequence of SEQ ID NO: 3. An isolated nucleic acid molecule comprising a nucleotide sequence which encodes the protein of the invention is furthermore provided, that is selected from the group consisting of a) a nucleic acid comprising a nucleotide sequence of SEQ ID NO: 1 or SEQ ID 2, b) a nucleic acid sequence having at least 80% identity to the nucleic acid sequence of SEQ ID NO: 1 or SEQ ID NO: 2 and c) a nucleic acid having a complementary sequence to the nucleic acid of a) or b).

“Clubroot” as used herein refers to the disease caused by the pathogen Plasmodiophora brassicae.

“Clubroot resistance” as used herein refers to resistance to one or more Plasmodiophora brassicae isolates, such as, but not limited to, resistance to the Plasmodiophora brassicae strain CR11 or CR6 corresponding to pathotype 5x (Yu et al., 2017, Sci Rep 7:4516). Said resistance refers to a reduction in damage caused by clubroot infection compared to damage caused on control plants. Damage can be assessed as, for example, formation of club-shaped galls on the roots, occurrence of wilting, stunting, yellowing, premature senescence etc. In particular, a reduction in damage is manifested in a reduced yield loss when plants are grown under disease pressure in the field, compared to control plants. Such reduction in yield loss can, for example, be due to the fact that the infection, reproduction, spread or survival of the pathogen is reduced or prevented in plants with enhanced resistance. Said resistance may also refer to plants that are completely resistant, i.e., plants on which no disease symptoms are found.

Clubroot resistance can be assessed using a scale from zero to three: zero: no clubbing, one: <25% of root system clubbed; two: 25 to 50% of root system clubbed; three: >50% of root system clubbed (Humpherson-Jones, 1989, Tests Agro Cult 10:36). The Disease Index (ID) can be calculated using the following equation:
[(#plants in class 0 * 0) + ([(#plants in class 1 * 1) + (#plants in class 2 * 2) + (#plants in class 3 * 3)] /total number of plants * 3

### (Strelkov et al., 2006, Can J Plant Pathol 28:467).

It is understood that environmental conditions, such as location, weather conditions and disease pressure, as well as individual perception of the person assessing disease symptoms, can have an effect on the scoring of clubroot resistance. Hence, variation in these factors in comparative tests should be minimized. Any other resistance ratings known in the art can be applied in accordance with this invention to compare the plants of the invention with control plants.

A “protein capable of conferring clubroot resistance” and the gene encoding the protein capable of conferring clubroot resistance, as used herein, are respectively a protein and a gene that confer resistance to a Plasmodiophora brassicae strain. A CRT resistance gene is present, for example, in Brassica napus cvs. Tosca. A “CRT clubroot resistance gene”, “CRT resistance gene” or “CRT gene” can be sufficient for resistance to Plasmodiophora brassicae strain CR11 or CR6 corresponding to pathotype 5x as described by Yu et al., 2017, Sci. Rep 7:4516. A “CRT clubroot resistance gene” or “CRT gene” can also be required together with another CRT clubroot resistance gene for resistance to Plasmodiophora brassicae strain CR11 or CR6 corresponding to pathotype 5x.

A CRT clubroot resistance gene, or CRT gene can encode a CRT amino acid sequence having at least 95%, or at least 98%, or at least 99%, or 100% sequence identity to SEQ ID NO: 3 and comprising the conserved domains described below. A CRT clubroot resistance gene, or

CRT gene, can comprise a nucleotide sequence having at least 80%, or at least 85%, or at least 90%, or at least 92%, or at least 95%, or at least 98%, or at least 99%, or 100% sequence identity to SEQ ID NO: 1 or to SEQ ID NO: 2 and comprise the nucleotide sequences encoding the conserved domains described below.

For the purpose of this invention, the “sequence identity” of two related nucleotide or amino acid sequences, expressed as a percentage, refers to the number of positions in the two optimally aligned sequences which have identical residues (x100) divided by the number of positions compared. A gap, i.e., a position in an alignment where a residue is present in one sequence but not in the other, is regarded as a position with non-identical residues. The “optimal alignment” of two sequences is found by aligning the two sequences over the entire length according to the Needleman and Wunsch global alignment algorithm (Needleman and Wunsch, 1970, J Mol Biol 48 (3): 443-53) in The European Molecular Biology Open Software Suite (EMBOSS, Rice et al., 2000, Trends in Genetics 16 (6): 276-277; see e.g. http://www.ebi.ac.uk/emboss/align/index.html) using default settings (gap opening penalty=10 (for nucleotides)/10 (for proteins) and gap extension penalty=0.5 (for nucleotides)/0.5 (for proteins)). For nucleotides the default scoring matrix used is EDNAFULL and for proteins the default scoring matrix is EBLOSUM62. It will be clear that whenever nucleotide sequences of RNA molecules are defined by reference to nucleotide sequence of corresponding DNA molecules, the thymine (T) in the nucleotide sequence should be replaced by uracil (U). Whether reference is made to RNA or DNA molecules will be clear from the context of the application.

The CRT protein according to the invention comprises a conserved Toll-Interleukin receptor domain (TIR-domain, from amino acid position 15 to amino acid position 149 of SEQ ID NO: 3), NB-ARC domain (from amino acid position 190 to amino acid position 453 on SEQ ID NO: 3; van der Biezen and Jones, 1998, Current Biology, Vol 8, 7: R226-R228), and a LRR domain (leucine rich repeat) with one or more LRR motifs (xxLxLxx) (from amino acid position 612 to amino acid position 867 of SEQ ID NO: 3).

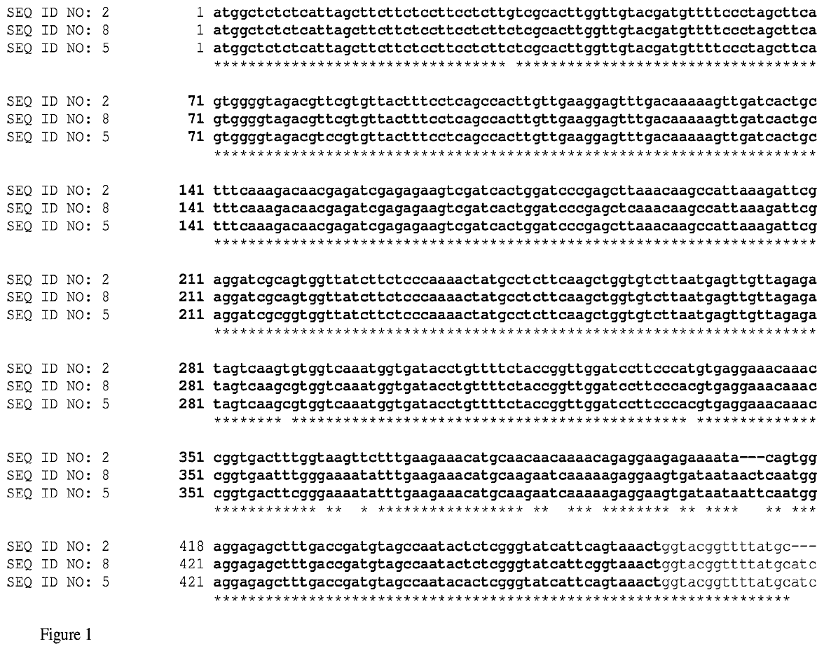

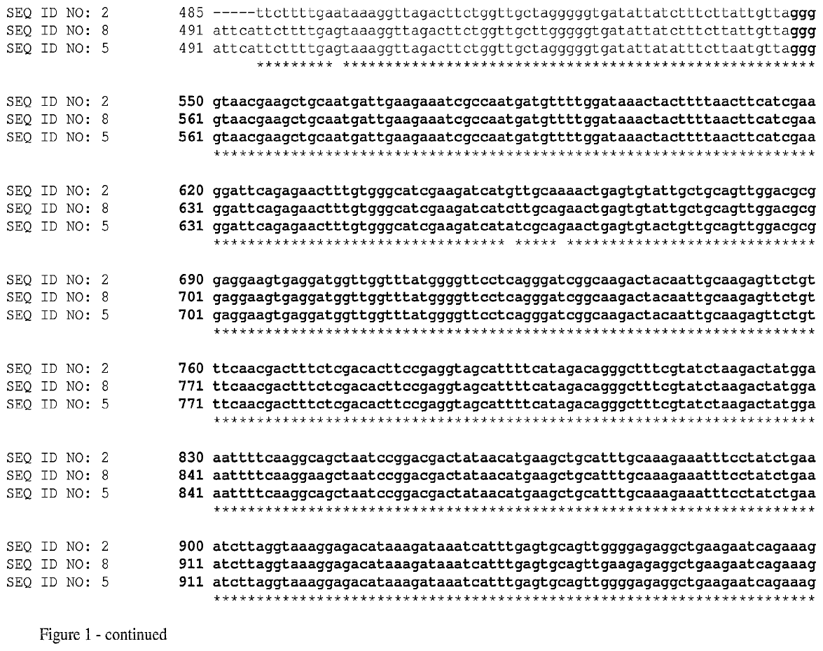

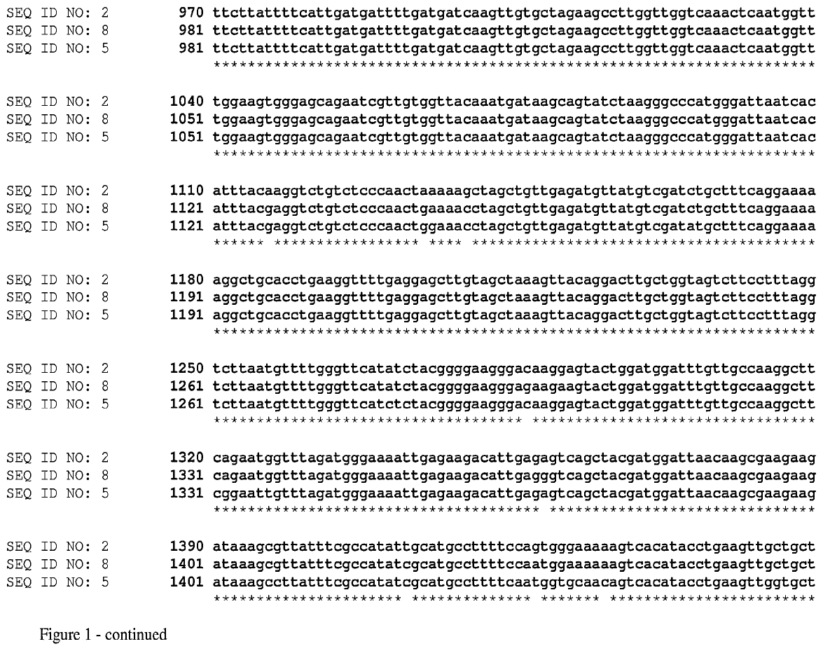

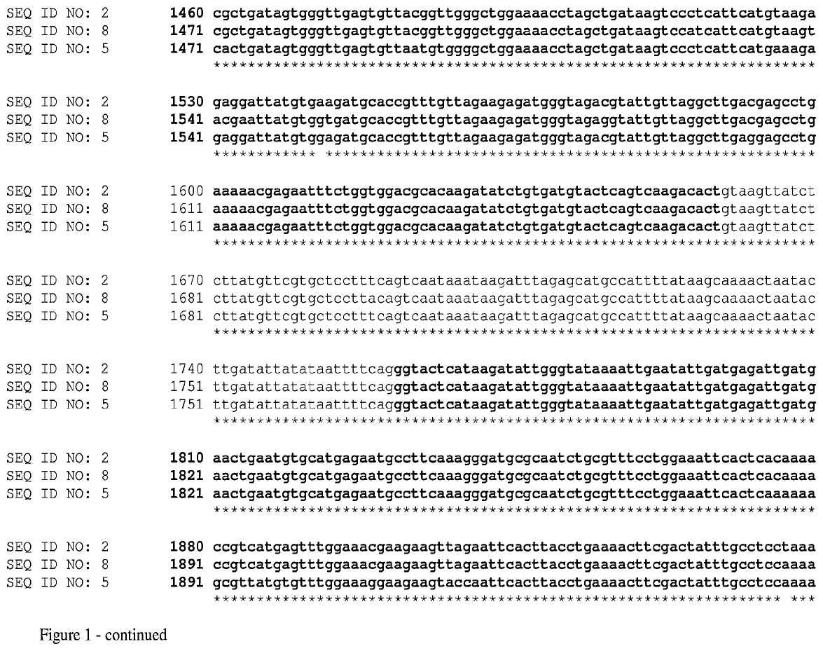

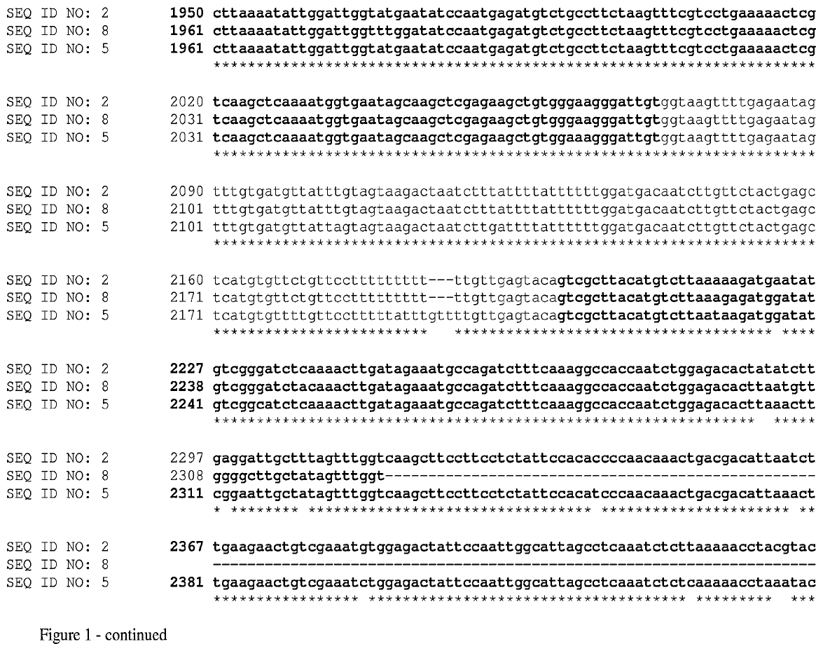

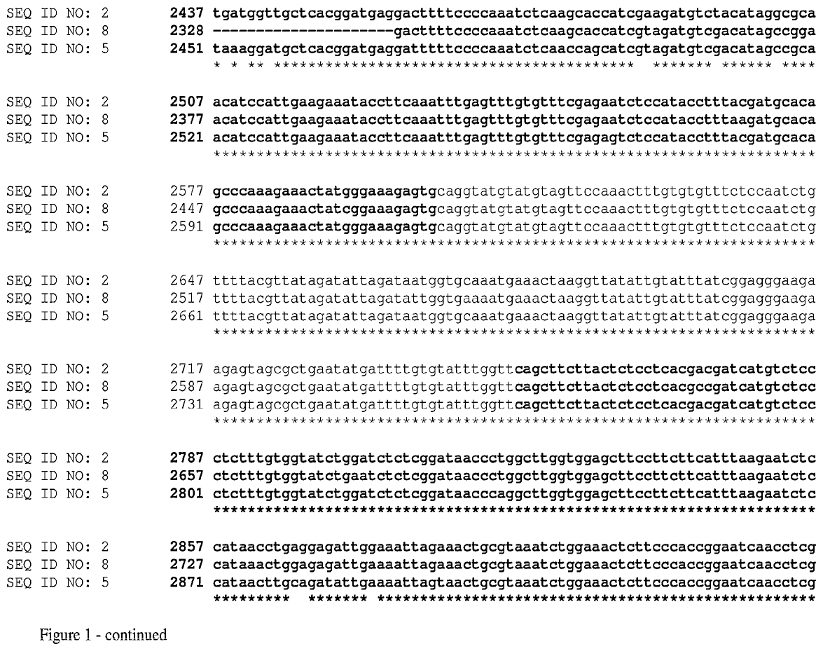

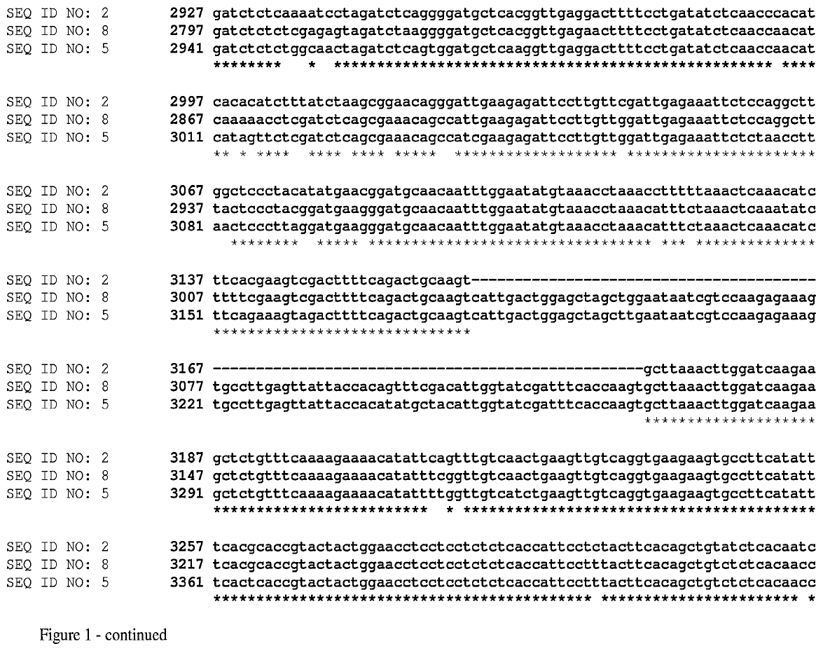

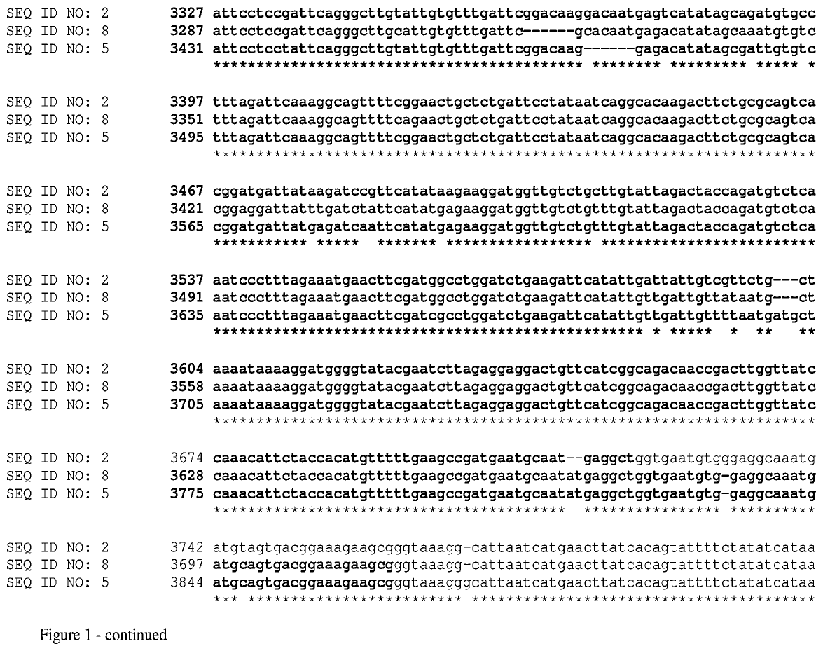

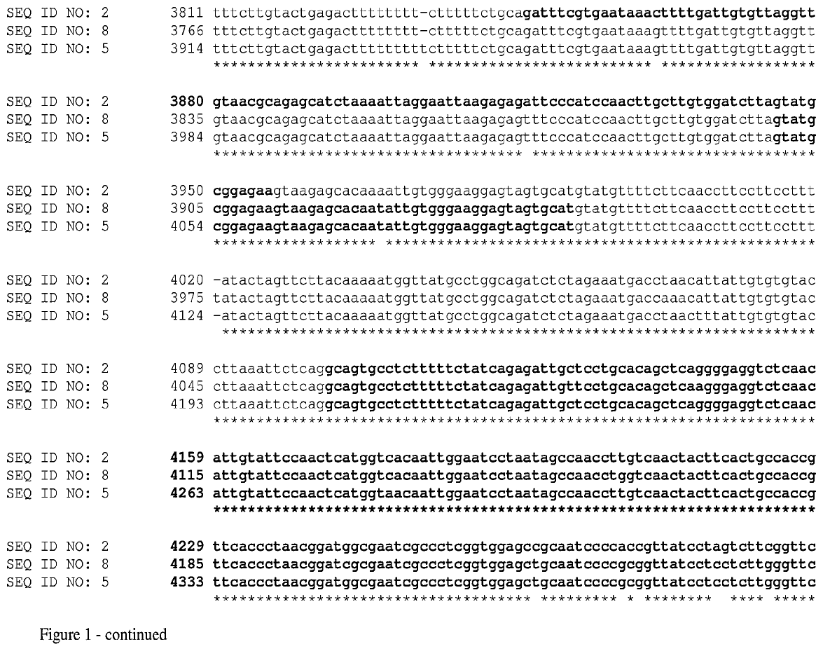

The conserved domains in the CRT protein according to the invention are shown in FIG. 1.

Furthermore, it is clear that variants of CRT proteins, wherein one or more amino acid residues have been deleted, substituted or inserted, can also be used to the same effect in the methods according to the invention, provided that the CRT domains are not affected by the deletion, substitution or insertion of amino-acid. These variant CRT proteins may have about 95% sequence identity to any one of the herein mentioned CRT proteins.

Examples of substitutions are the conservative substitutions, i.e. substitutions of one amino-acid by another having similar physiochemical properties. These substitutions are known not to affect the structure of a protein. Such substitutions are achieved by replacing one aminoacid by another aminoacid belonging to the same group as follows:
Group 1: Cysteine (C);
Group 2: Phenylalanine (F), Tryptophan (W) and Tyrosine (Y);
Group 3: Histidine (H), Lysing K) and Arginine (R);
Group 4: Aspartic acid (D), Glutamic acid (E), Asparagine (N) and Glutamine (Q);
Group 5: Isoleucine (I), Leucine (L), Methionine (M) and Valine (V);
Group 6: Alanine (A), Glycine (G), Proline (P), Serine(S) and Threonine (T).

“Isolated nucleic acid” or “Isolated DNA” as used herein refers to DNA not occurring in its natural genomic context, irrespective of its length and sequence. Isolated DNA can, for example, refer to DNA which is physically separated from the genomic context, such as a fragment of genomic DNA. Isolated DNA can also be an artificially produced DNA, such as a chemically synthesized DNA, or such as DNA produced via amplification reactions, such as polymerase chain reaction (PCR) well-known in the art. Isolated DNA can further refer to DNA present in a context of DNA in which it does not occur naturally. For example, isolated DNA can refer to a piece of DNA present in a plasmid. Further, the isolated DNA can refer to a piece of DNA present in another chromosomal context than the context in which it occurs naturally, such as for example at another position in the genome than the natural position, in the genome of another species than the species in which it occurs naturally, or in an artificial chromosome.

It is another object of the present invention to provide a recombinant gene comprising a plant expressible promoter operably linked to a nucleic acid sequence encoding the protein of the invention and optionally, a transcription termination and polyadenylation sequence, preferably a transcription termination and polyadenylation region functional in plants. In another object, said nucleic acid is selected from the group consisting of a) a nucleic acid comprising a nucleotide sequence of SEQ ID NO: 1 or SEQ ID NO: 2; b) a nucleic acid sequence having at least 80% identity to the nucleic acid sequence of SEQ ID NO: 1 or SEQ ID

NO: 2; and c) a nucleic acid having a complementary sequence to the nucleic acid of a) or b). In another embodiment, said plant expressible promoter is selected from the group consisting of constitutive promoter, inducible promoter, tissue specific promoter. The plant expressible promoter may be the CaMV35S promoter.

As used herein a “recombinant gene” refers to a nucleic acid construct which is not normally found in a plant species. A recombinant nucleic acid construct can be DNA or RNA. “Recombinant DNA construct” and “recombinant gene” are used interchangeably to denote a gene in which the promoter or one or more other regulatory regions of the gene are not associated in nature with part or all of the transcribed DNA region, or a gene which is present in a locus in the plant genome in which it does not occur naturally.

The phrase “operably linked” refers to the functional spatial arrangement of two or more nucleic acid regions or nucleic acid sequences. For example, a promoter region may be positioned relative to a nucleic acid sequence such that transcription of a nucleic acid sequence is directed by the promoter region. Thus, a promoter region is “operably linked” to the nucleic acid sequence. “Functionally linked” is an equivalent term.

As used herein, the term “plant-expressible promoter” means a DNA sequence that is capable of controlling (initiating) transcription in a plant cell. This includes any promoter of plant origin, but also any promoter of non-plant origin which is capable of directing transcription in a plant cell, i.e., certain promoters of viral or bacterial origin such as the CaMV35S (Harpster et al. (1988) Mol Gen Genet. 212 (1): 182-90, the subterranean clover virus promoter No 4 or No 7 (WO9606932), or T-DNA gene promoters but also tissue-specific or organ-specific promoters including but not limited to seed-specific promoters (e.g., WO89/03887), organ-primordia specific promoters (An et al. (1996) Plant Cell 8 (1): 15-30), stem-specific promoters (Keller et al., (1988) EMBO J. 7 (12): 3625-3633), leaf specific promoters (Hudspeth et al. (1989) Plant Mol Biol. 12:579-589), mesophyl-specific promoters (such as the light-inducible Rubisco promoters), root-specific promoters (Keller et al. (1989) Genes Dev. 3:1639-1646), tuber-specific promoters (Keil et al. (1989) EMBO J. 8 (5): 1323-1330), vascular tissue specific promoters (Peleman et al. (1989) Gene 84:359-369), stamen-selective promoters (WO 89/10396, WO 92/13956), dehiscence zone specific promoters (WO 97/13865) and the like.

Suitable promoters for the invention are constitutive plant-expressible promoters. Constitutive plant-expressible promoters are well known in the art, and include the CaMV35S promoter (Harpster et al. (1988) Mol Gen Genet. 212 (1): 182-90), Actin promoters, such as, for example, the promoter from the Rice Actin gene (McElroy et al., 1990, Plant Cell 2:163), the promoter of the Cassava Vein Mosaic Virus (Verdaguer et al., 1996 Plant Mol. Biol. 31:1129), the GOS promoter (de Pater et al., 1992, Plant J. 2:837), the Histone H3 promoter (Chaubet et al., 1986, Plant Mol Biol 6:253), the Agrobacterium tumefaciens Nopaline Synthase (Nos) promoter (Depicker et al., 1982, J. Mol. Appl. Genet. 1:561), or Ubiquitin promoters, such as, for example, the promoter of the maize Ubiquitin-1 gene (Christensen et al., 1992, Plant Mol. Biol. 18:675).

A further promoter suitable for the invention is the endogenous promoter driving expression of the gene encoding an CRT protein.

A “transcription termination and polyadenylation region” as used herein is a sequence that drives the cleavage of the nascent RNA, whereafter a poly (A) tail is added at the resulting RNA 3′ end, functional in plant cells. Transcription termination and polyadenylation signals functional in plant cells include, but are not limited to, 3′nos, 3′35S, 3′his and 3′g7.

The present invention furthermore provides a plant or plant cell and a seed comprising

the recombinant gene according to the invention. The present invention also provides a plant or plant cell and a seed comprising a heterologous CRT gene encoding the protein of the invention but not comprising at least one of the sequences of SEQ ID NOs: 11 and 12. The described plants may be a Brassicacae, and may further comprise at least one other disease resistance gene selected from the group consisting of a clubroot resistance gene, a blackleg resistance gene, a

Sclerotinia resistance gene, a Verticillium resistance gene, a Fusarium wilt resistance gene, an Aster Yellows resistance gene, an Alternaria resistance gene, and a Grey Stem resistance gene.

“Brassicaceae” or “Brassicaceae plant” as used herein refers to plants belonging to the family of Brassicaceae plants, also called Cruciferae or mustard family. Examples of Brassicaceae are, but are not limited to, Brassica species, such as Brassica napus, Brassica oleracea, Brassica rapa, Brassica carinata, Brassica nigra, and Brassica juncea; Raphanus species, such as Raphanus caudatus, Raphanus raphanistrum, and Raphanus sativus; Matthiola species; Cheiranthus species; Camelina species, such as Camelina sativa; Crambe species, such as Crambe abyssinica and Crambe hispanica; Eruca species, such as Eruca vesicaria; Sinapis species such as Sinapis alba; Diplotaxis species; Lepidium species; Nasturtium species; Orychophragmus species; Armoracia species, Eutrema species; Lepidium species; and Arabidopsis species.

A “Brassica plant” refers to allotetraploid or amphidiploid Brassica napus (AACC. 2n=38), Brassica juncea (AABB, 2n=36), Brassica carinata (BBCC, 2n=34), or to diploid Brassica rapa (syn. B. campestris) (AA. 2n=20), Brassica oleracea (CC, 2n=18) or Brassica nigra (BB, 2n=16).

The invention further provides a method for obtaining a clubroot resistant Brassicacae plant, comprising a) introducing or providing the clubroot resistance gene encoding the protein according to the invention to a Brassicacae plant cell, to create a Brassicacae cell, and b) regenerating a plant from said cell. Said Brassicacae plant may be selected from the group consisting of Brassica napus, Brassica juncea, Brassica oleracea, Brassica rapa, Brassica nigra and Brassica carinata. In another embodiment, the clubroot resistance gene is introduced or provided to the Brassicacae plant cell by providing or introducing to the Brassicacae plant cell the recombinant gene according to the invention. In yet another embodiment, the clubroot resistance gene is introduced or provided to the Brassicacae plant cell by a) providing a first Brassica plant that comprises the clubroot resistance sequence according to the invention; b) providing a second Brassica plant that lacks the clubroot resistance sequence of the invention; c) crossing the first Brassica plant with the second Brassica plant to provide progeny Brassica plant; and d) selecting Brassica progeny plant that tests positive for the presence of the clubroot resistance sequence according to the invention as being Brassica plant into which the clubroot resistance sequence of the invention has been introduced.

A transgene can be provided to a plant or plant cell using methods well-known in the art. Methods for introduction of genes into plant cells to create transgenic plants are not deemed critical for the current invention and any method to provide plant cells with a transgene suitable for a particular plant species can be used. Such methods are well known in the art and include Agrobacterium-mediated transformation, particle gun delivery, microinjection, electroporation of intact cells, polyethyleneglycol-mediated protoplast transformation, electroporation of protoplasts, liposome-mediated transformation, silicon-whiskers mediated transformation etc.

Said transgene may be stably integrated into the genome of said plant cell, resulting in a transformed plant cell. The transformed plant cells obtained in this way may then be regenerated into mature fertile transformed plants.

A “molecular marker”, or a “marker”, as used herein, refers to a polymorphic locus,

i.e. a polymorphic nucleotide (a so-called single nucleotide polymorphism or SNP) or a polymorphic DNA sequence (which can be insertion or deletion of a specific DNA sequence at a specific locus, or polymorphic DNA sequences). A marker refers to a measurable, genetic characteristic with a fixed position in the genome, which is normally inherited in a Mendelian fashion, and which can be used for mapping of a trait of interest. Thus, a molecular marker may be a short DNA sequence, such as a sequence surrounding a single base-pair change, i.e. a single nucleotide polymorphism or SNP, or a long DNA sequence, such as microsatellites or Simple Sequence Repeats (SSRs). The nature of the marker is dependent on the molecular analysis used and can be detected at the DNA, RNA or protein level. Genetic mapping can be performed using molecular markers such as, but not limited to, RFLP (restriction fragment length polymorphisms; Botstein et al. (1980), Am J Hum Genet 32:314-331; Tanksley et al. (1989), Bio/Technology 7:257-263), RAPD [random amplified polymorphic DNA; Williams et al. (1990), NAR 18:6531-6535], AFLP [Amplified Fragment Length Polymorphism; Vos et al. (1995) NAR 23:4407-4414], SSRs or microsatellites [Tautz et al. (1989), NAR 17:6463-6471].Appropriate primers or probes are dictated by the mapping method used.

The term “AFLPR” (AFLPR is a registered trademark of KeyGene N.V., Wageningen, The Netherlands), “AFLP analysis” and “AFLP marker” is used according to standard terminology [Vos et al. (1995), NAR 23:4407-4414; EP0534858;http://www.keygene.com/keygene/techs-apps/]. Briefly, AFLP analysis is a DNA fingerprinting technique which detects multiple DNA restriction fragments by means of PCR amplification.

The AFLP technology usually comprises the following steps: (i) the restriction of the DNA with two restriction enzymes, preferably a hexa-cutter and a tetra-cutter, such as EcoRI, Pstl and Msel; (ii) the ligation of double-stranded adapters to the ends of the restriction fragments, such as EcoRI, Pstl and Msel adaptors; (iii) the amplification of a subset of the restriction fragments using two primers complementary to the adapter and restriction site sequences, and extended at their 3′ ends by one to three “selective” nucleotides, i.e., the selective amplification is achieved by the use of primers that extend into the restriction fragments, amplifying only those fragments in which the primer extensions match the nucleotides flanking the restriction sites. AFLP primers thus have a specific sequence and each AFLP primer has a specific code (the primer codes and their sequences can be found at the Keygene website: http://www.keygene.com/keygene/pdf/PRIMERCO.pdf; herein incorporated by reference); (iv) gel electrophoresis of the amplified restriction fragments on denaturing slab gels or cappilaries; (v) the visualization of the DNA fingerprints by means of autoradiography, phosphor-imaging, or other methods. Using this method, sets of restriction fragments may be visualized by PCR without knowledge of nucleotide sequence. An AFLP marker, as used herein, is a DNA fragment of a specific size, which is generated and visualized as a band on a gel by carrying out an AFLP analysis. Each AFLP marker is designated by the primer combination used to amplify it, followed by the approximate size (in base pairs) of the amplified DNA fragment. It is understood that the size of these fragments may vary slightly depending on laboratory conditions and equipment used. Every time reference is made herein to an AFLP marker by referring to a primer combination and the specific size of a fragment, it is to be understood that such size is approximate, and comprises or is intended to include the slight variations observed in different labs. Each AFLP marker represents a certain locus in the genome.

The term “SSR” refers to Simple Sequence Repeats or microsatellite [Tautz et al. (1989), NAR 17:6463-6471]. Short Simple Sequence stretches occur as highly repetitive elements in all eukaryotic genomes. Simple sequence loci usually show extensive length polymorphisms. These simple sequence length polymorphisms (SSLP) can be detected by polymerase chain reaction (PCR) analysis and be used for identity testing, population studies, linkage analysis and genome mapping.

It is understood that molecular markers can be converted into other types of molecular markers. When referring to a specific molecular marker in the present invention, it is understood that the definition encompasses other types of molecular markers used to detect the genetic variation originally identified by the specific molecular markers. For example, if an AFLP marker is converted into another molecular marker using known methods, this other marker is included in the definition. For example, AFLP markers can be converted into sequence-specific markers such as, but not limited to STS (sequenced-tagged-site) or SCAR (sequence-characterized-amplified-region) markers using standard technology as described in Meksem et al. [(2001), Mol Gen Genomics 265 (2): 207-214], Negi et al. [(2000), TAG 101:146-152], Barret et al. (1989), TAG 97:828-833], Xu et al. [(2001), Genome 44 (1): 63-70], Dussel et al. [(2002), TAG 105:1190-1195] or Guo et al. [(2003), TAG 103:1011-1017]. For example, Dussel et al. [(2002), TAG 105:1190-1195] converted AFLP markers linked to resistance into PCR-based sequence tagged site markers such as indel (insertion/deletion) markers and CAPS (cleaved amplified polymorphic sequence) markers.

Suitable molecular markers are, for example SNP markers (Single Nucleotide Polymorphisms), AFLP markers, microsatellites, minisatellites, Random Amplified Polymorphic DNA's (RAPD) markers, RFLP markers, Sequence Characterized Amplified Regions (SCAR) markers, and others, such as TRAP markers described by Hu et al. 2007, Genet Resour Crop Evol 54:1667-1674).

Methods and assays for marker detection, or for analyzing the genomic DNA for the presence of a marker, are widely known in the art. The presence of a marker can, for example be detected in hybridization-based methods (e.g. allele-specific hybridization), using Taqman, PCR-based methods, oligonucleotide ligation based methods, or sequencing-based methods.

A useful assay for detection of SNP markers is for example KBioscience Competitive Allele-Specific PCR. For developing the KASP-assay 70 base pairs upstream and 70 basepairs downstream of the SNP are selected and two allele-specific forward primers and one allele specific reverse primer is designed. See e.g. Allen et al. 2011, Plant Biotechnology J. 9, 1086-1099,especially p1097-1098 for KASP assay method (incorporated herein by reference).

A “molecular marker linked to the CRT clubroot resistance gene”, or a “molecular marker linked to the presence of the CRT clubroot resistance gene” as used herein refers to a molecular marker in a region in the genome that inherits with the CRT clubroot resistance gene as a single genetic unit in at least 50% of the cases. Thus, in this respect, the term linked can be a separation of about 50 cM, or less such as about 40 cM, about 30 CM, about 20 cM, about 10cM, about 7.5 CM, about 6 cM, about 5 CM, about 4 cM, about 3 cM, about 2.5 CM, about 2 cM, or even less. Said “molecular marker linked to the CRT clubroot resistance gene” is thus a marker which is linked to the CRT clubroot resistance gene. Said marker can be based on the CRT clubroot resistance gene itself, such as presence or absence of the CRT clubroot resistance gene.

Suitable are markers that are linked to the CRT clubroot resistance gene can be developed using methods known in the art. New markers suitable for the invention can be developed based on the CRT sequence. It is understood that such markers can be developed by comparing the sequence of the CRT clubroot resistance gene from the resistant Brassicaceae line with the sequence of the same gene in a susceptible Brassicaceae line; identifying a specific sequence region in the CRT clubroot resistance gene which does not occur in the corresponding gene of the susceptible Brassicaceae line. A molecular marker linked to the CRT clubroot resistance gene can thus be a marker detecting the presence of the CRT clubroot resistance gene. or can be a marker directly detecting the presence of the sequence of SEQ ID NOs: 1 or 2. A molecular marker linked to the CRT clubroot resistance gene can also be a marker in the sequences flanking the CRT clubroot resistance gene, which is polymorphic between lines comprising the CRT clubroot resistance gene and lines lacking, but which inherits with the CRT clubroot resistance gene as a single genetic unit in at least 50% of the cases.

The absence of the CRT clubroot resistance gene can be determined by the absence of marker alleles that are linked to the presence of the CRT clubroot resistance gene (CRT clubroot resistance marker alleles. Furthermore, markers suitable to determine the absence of the CRT clubroot resistance gene can be marker alleles which are linked to the CRS clubroot susceptibility gene.

Analysis for the presence of markers according to the invention can be performed with a first primer and a second primer, and, optionally, a probe, selected from the group consisting of a first primer consisting of a sequence of 15 to 30 nucleotides, or 15 to 25nucleotides, or 18 to 22 nucleotides of the CRT clubroot resistance genes according to the invention, a second primer being complementary to a sequence of 15 to 30 nucleotides, or 15 to 25 nucleotides, or 18 to 22 nucleotides of the CRT clubroot resistance genes according to the invention, and wherein the distance between said first and said second primer on the CRT clubroot resistance gene is between 1 and 400 bases, or between 1 and 150 bases, and wherein the first primer is located, with respect to the CRT coding sequence, upstream of said second primer, and a probe which is identical to at least 15 nucleotides, or at least 18 nucleotides, but not more than 25 nucleotides, or not more than 22 nucleotides of the sequence of the CRT clubroot resistance gene between said first and said second primer, provided that either the sequence of the first primer, or the sequence of the second primer, or the sequence of said probe is not present in the corresponding locus in a susceptible Brassicaceae plant. Said probe may be labelled, such as, for example, described in U.S. Pat. No. 5,538,848.

Analysis for the presence of markers according to the invention can be performed with a first and second primer as described above recognizing both the CRT sequence and the corresponding gene in the susceptible Brassicaceae line, a first probe recognizing a sequence of the CRT clubroot resistance gene as described above, but not recognizing a sequence between said first and said second primer in the susceptible Brassicaceaea line, and a second probe recognizing a sequence between said first and said second primer in the susceptible Brassicaceaea line, but not of the CRT clubroot resistance gene, and wherein said the label of the first probe is different from that of the second probe.

Further suitable primers for analysis of the presence of markers according to the invention are markers a first primer as described above recognizing both the CRT sequence and the corresponding gene in the susceptible Brassicaceae line, a second primer recognizing the CRT sequence but not the corresponding gene in the susceptible Brassicaceae line, and a third primer recognizing the corresponding locus in the susceptible Brassicaceae line but not the CRT sequence. Said second and third primer may be labelled as indicated above, and said second primer may contain a label which is different from said third primer.

Identification of PCR products specific for the CRT clubroot resistance genes and for the corresponding gene in the susceptible Brassicaceae line can occur e.g. by size estimation after gel or capillary electrophoresis (e.g. for the CRT clubroot resistance gene and for the corresponding gene in the susceptible Brassicaceae line comprising a number of inserted or deleted nucleotides which results in a size difference between the fragments amplified from the CRT clubroot resistance gene and for the corresponding gene in the susceptible Brassicaceae, such that said fragments can be visibly separated on a gel); by evaluating the presence or absence of the two different fragments after gel or capillary electrophoresis, whereby the diagnostic PCR amplification of the CRT clubroot resistance gene can, optionally, be performed separately from the diagnostic PCR amplification of the corresponding gene in the susceptible line; by direct sequencing of the amplified fragments; or by fluorescence-based detection methods.

“Introducing” or “providing” in connection with the present application may relate to the placing of genetic information in a plant cell or plant by artificial means. This can be effected by any method known in the art for introducing RNA or DNA into plant cells, tissues, protoplasts or whole plants.

It is another object of the invention to provide a Brassicacae plant obtained by the method according to the invention. In a further aspect, the Brassicacae plant comprises the clubroot resistance sequence according to the invention and at least one other disease resistance gene, said other disease resistance gene selected from the group consisting of a clubroot resistance gene, a blackleg resistance gene, a Sclerotinia resistance gene, a Verticillium resistance gene, a Fusarium wilt resistance gene, an Aster Yellows resistance gene, an Alternaria resistance gene, and a Grey Stem resistance gene. Such Brassicacae plant may be a Brassica napus. Seeds of this Brassicacae plant are also provided and they may be hybrid seeds. With such hybrid seeds, being Brassica napus hybrid seeds, and developing into plants, the solid component of the seeds may contain less than 30 micromoles of any one or any mixture of 3-butenyl glucosinolate, 4-pentenyl glucosinolate, 3-hydroxy-3 butenyl glucosinolate, and 2-hydroxy-4-pentenyl glucosinolate per gram of air-dry, oil-free solid.

Said clubroot resistance gene may be a Crr2, Crr4, Crr3, CRk, CRc. CR2a, CR2b, pb-3.pb-4, Pb-Bol, Pb-Bo2, Pb-Bo3, Pb-Bo4, Pb-BoSa, Pb-Bo5b, Pb-Bo8, Pb-Bo9a, Pb-Bo9b, Pb-Bn1,PbBn-01:60-1, PbBn-01:60-2, PbBn-01:60-3, PbBn-01:60-4, PbBn-01:07-1, PbBn-01:07-2,PbBn-01:07-3., PbBn-e4x04-1. PbBn-a-1. PbBn-1-1, PbBn-1-2, PbBn-k-1, PbBn-k-2. PbBn-k-3,PbBn-Korp-1, PbBn-Korp-2, PbBn-Korp-3, PbBn-Korp-4, PbBn-Korp-5 as described by

Piao et al., 2009, supra, or may be a CRa gene as described by Ueno et al., 2012, supra, a Crr1gene as described by Hatakeyama et al., 2013, supra and in WO2012/039445, or a CRb gene as described by Kato et al., 2013, supra, a CRL gene as described in WO2017/102923.

Said Blackleg resistance gene may, for example, be BLMRI and BLMR2 (WO 2011/044694), LepR3 (Larkan et al., 2013, New Phytol 197:595 and WO 2008/101343), or

Lem-08-syl (EP 1547462 and US 2005/0142122). Said Sclerotinia resistance gene may be a sclerotinia resistance gene as described in WO 2005/090578.

Said other disease resistance gene may be present in their native chromosomal position. For example, said other disease resistance genes can be introduced by introgression in the plant according to the invention from the cultivar or-species from which they are derived.

A plant which is clubroot resistant refers to a plant assessed at scale zero or one upon natural infection with the clubroot pathogen, or to a plant assessed at scale zero, one or two upon natural infection with the clubroot pathogen. A clubroot resistant population is a population with a disease index (ID) of less than 30%. A plant with increased clubroot resistance is a plant in which the percentage of the root system which is clubbed is decreased with at least 5%, or at least 10%, or at least 15%, or at least 20%, or at least 30%, or at least 40%, or at least 50%, or at least 70%, or at least 95%, or with 100%, i.e. no clubbing, or refers to a population of plants in which the disease index is reduced with at least 3%, or at least 5%, or at least 8%, or at least 10%, or at least 15%, or at least 20%, or at least 30%, or at least 40%, or at least 50%, or at least 70%, or at least 95%, or with 100%, i.e. all plants of the population are classified in class 0 (no clubbing).

Hybrid seeds of the plants according to the invention may be generated by crossing two inbred parental lines, wherein at least one of the inbred parental lines comprises the CRT clubroot resistance genes according to the invention. In order to produce pure hybrid seeds one of the parental lines is male sterile and is pollinated with pollen of the other line. By growing parental lines in rows and only harvesting the Fl seed of the male sterile parent, pure hybrid seeds are produced. To generate male sterile parental lines, the system as described in EP 0,344,029 or U.S. Pat. No. 6,509,516 may be used, wherein a gene encoding a phytotoxic protein (barnase) is expressed under the control of a tapetum specific promoter, such as TA29, ensuring selective destruction of tapetum cells. Transformation of plants with the chimeric gene pTA29: barnase results in plants in which pollen formation is completely prevented [Mariani et al. (1990), Nature 347:737-741]. Cytochemical and histochemical analysis of anther development of Brassica napus plants comprising the chimeric pTA29-barnase gene is described by De Block and De Brouwer [(1993), Planta 189:218-225]. To restore fertility in the progeny of a male-sterile plant the male-sterile plant (MS parent) is crossed with a transgenic plant (RF parent) carrying a fertility-restorer gene, which when expressed is capable of inhibiting or preventing the activity of the male-sterility gene [U.S. Pat. Nos. 5,689,041; 5,792,929; De Block and De Brouwer, supra]. The use of co-regulating genes in the production of male-sterile plants to increase the frequency of transformants having good agronomical performance is described in WO96/26283. Typically, when the sterility DNA encodes a barnase, the co-regulating DNA will encode a barstar, preferably an optimized barstar gene is used as described in published PCT patent application WO 98/10081. It is understood that different promoters may be used to drive barnase expression in order to render the plant male sterile. Likewise, barstar may be operably linked to different promoters, such as 35S from Cauliflower mosaic virus.

Male sterile plants can also be generated using other techniques, such as cytoplasmic male sterility/restorer systems [e.g. the Ogura system, published U.S. patent application Ser. No. 20/020,032916, U.S. Pat. No. 6,229,072, WO97/02737, U.S. Pat. No. 5,789,566 or the Polima system of U.S. Pat. No. 6,365,798, WO98/54340 or the Kosena system of WO95/09910, U.S. Pat. No. 5,644,066].

Either the MS parent or the RF parent, or both, may comprise the CRT clubroot resistance genes according to the invention. This can be accomplished by either introducing the CRT clubroot resistance genes into an elite B. napus line and then transforming this line with pTA29-barnase or with pNOS-barstar using known methods. Alternatively the CRT clubroot resistance genes can be introduced directly into a transgenic MS or RF parent line, by crossing a plant comprising the CRT clubroot resistance genes with the MS parent or RF-parent, or by transformation of the MS parent or the RF parent. The F1 hybrid seeds generated from the cross between the MS and RF parent will then contain the CRT clubroot resistance genes.

Further embodiments disclose a kit for the detection of a CRT clubroot resistance locus in Brassica DNA samples, wherein said kit comprises one or more PCR primer pairs, which are able to amplify a DNA marker linked to CRT. The disclosed kit may comprise two primers recognizing CRT and not recognizing the nucleotide sequence of SEQ ID NOs: 4, 5, 7 or 8.

A “kit”, as used herein, refers to a set of reagents for the purpose of performing the method of the invention, more particularly, the identification of the CRT clubroot resistance genes in biological samples or the determination of the zygosity status of plant material comprising the CRT clubroot resistance genes. More particularly, a preferred embodiment of the kit of the invention comprises at least two specific primers for identification of the CRT clubroot resistance genes, or at least two or three specific primers for the determination of the zygosity status. Optionally, the kit can further comprise any other reagent. Alternatively, according to another embodiment of this invention, the kit can comprise at least one specific probe, which specifically hybridizes with nucleic acid of biological samples to identify the presence of the CRT clubroot resistance genes therein, or at least two or three specific probes for the determination of the zygosity status. Optionally, the kit can further comprise any other reagent (such as but not limited to hybridizing buffer, label) for identification of the CRT clubroot resistance genes in biological samples, using the specific probe.

The kit of the invention can be used, and its components can be specifically adjusted, for purposes of quality control (e.g., purity of seed lots), detection of the presence or absence of the CRT clubroot resistance gene in plant material or material comprising or derived from plant material, such as but not limited to food or feed products. The zygosity status of the CRT clubroot resistance gene can be determined by using alternative sets of primers and/or probes that specifically the CRT gene and the corresponding gene in a susceptible Brassicaceae line.

The term “primer” as used herein encompasses any nucleic acid that is capable of priming the synthesis of a nascent nucleic acid in a template-dependent process, such as PCR. Typically, primers are oligonucleotides from 10 to 30 nucleotides, but longer sequences can be employed. Primers may be provided in double-stranded form, though the single-stranded form is preferred. Probes can be used as primers, but are designed to bind to the target DNA or RNA and need not be used in an amplification process.

The term “recognizing” as used herein when referring to specific primers, refers to the fact that the specific primers specifically hybridize to a specific nucleic acid sequence under the conditions set forth in the method (such as the conditions of the PCR identification protocol), whereby the specificity is determined by the presence of positive and negative controls. It is standard in the art to design primers recognizing specifically the nucleotide sequence of a given allele of a gene.

Yet another embodiment provides a method of producing food, feed, or an industrial product comprising obtaining the plant according to the invention or a part thereof, and preparing the food, feed or industrial product from this plant or part thereof. In a further object, said food or feed is oil, meal, grain, starch, flour or protein; or said industrial product is biofuel, fiber, industrial chemicals, a pharmaceutical or a nutraceutical.

Further provided is the use of the nucleotide sequence of SEQ ID NOs: 1 or 2 or of the amino acid sequence of SEQ ID NO: 3 to identify homologous clubroot resistance genes.

Homologous clubroot resistance genes can be identified using methods known in the art. Homologous nucleotide sequence may be identified and isolated by hybridization under stringent conditions using as probes a nucleic acid comprising the nucleotide sequence of SEQ

ID NOs: 1 or 2 or part thereof. Other sequences encoding CRT may also be obtained by DNA amplification using oligonucleotides specific for genes encoding CRT as primers, such as but not limited to oligonucleotides comprising or consisting of about 20 to about 50 consecutive nucleotides from SEQ SEQ ID NOs: 1 or 2 or its complement. Homologous clubroot resistance genes can be identified in silico using Basic Local Alignment Search Tool (BLAST) homology search with other nucleotide or amino acid sequences. Functionality of the identified homologous clubroot resistance genes can be validated using the methods described herein, such as transforming a the clubroot resistance gene under control of a plant-expressible promoter in a plant not being clubroot resistant.

Hybridization occurs when the two nucleic acid molecules anneal to one another under appropriate conditions. Nucleic acid hybridization is a technique well known to those of skill in the art of DNA manipulation. The hybridization property of a given pair of nucleic acids is an indication of their similarity or identity. Another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence. “Stringent hybridization conditions” and “stringent hybridization wash conditions” in the context of nucleic acid hybridization experiments such as Southern and Northern hybridization are sequence dependent, and are different under different environmental parameters. An example of highly stringent wash conditions is 0.15 M NaCI at 72° C. for about 15 minutes. An example of stringent wash conditions is a 0.2 X SSC wash at 65° C. for 15minutes. Often, a high stringency wash is preceded by a low stringency wash to remove background probe signal. An example medium stringency wash for a duplex of, e.g., more than 100 nucleotides, is 1 X SSC at 45° C. for 15 minutes. An example low stringency wash for a duplex of, e.g., more than 100 nucleotides, is 4 to 6 X SSC at 40° C. for 15 minutes. For short probes (e.g., about 10 to 50 nucleotides), stringent conditions typically involve salt concentrations of less than about 1.5 M. more preferably about 0.01 to 1.0 M, Na ion concentration (or other salts) at pH 7.0 to 8.3, and the temperature is typically at least about 30° C. and at least about 60° C. for long probes (e.g., >50 nucleotides). Stringent conditions may also be achieved with the addition of destabilizing agents such as formamide. In general, a signal to noise ratio of 2 X (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code. Very stringent conditions are selected to be equal to the Tm for a particular probe. An example of stringent conditions for hybridization of complementary nucleic acids which have more than 100complementary residues on a filter in a Southern or Northern blot is 50% formamide, e.g., hybridization in 50% formamide, 1 M NaCI, 1% SDS at 37° C., and a wash in 0.1 x SSC at 60 to 65° C. Exemplary low stringency conditions include hybridization with a buffer solution of 30 to 35% formamide, 1 M NaCI, 1% SDS (sodium dodecyl sulphate) at 37° C., and a wash in 1 X to 2X SSC (20 X SSC=3.0 M NaCI/0.3 M trisodium citrate) at 50 to 55° C. Exemplary moderate stringency conditions include hybridization in 40 to 45% formamide, 1.0 M NaCI, 1% SDS at 37° C., and a wash in 0.5 X to 1 X SSC at 55 to 60° C. The following are examples of sets of hybridization/wash conditions that may be used to clone orthologous nucleotide sequences that are substantially identical to reference nucleotide sequences of the present invention: a reference nucleotide sequence preferably hybridizes to the reference nucleotide sequence in 7% sodium dodecyl sulfate (SDS), 0.5 M NaPO₄, 1 mM EDTA at 50° C. with washing in 2 X SSC, 0.1% SDS at 50° C. more desirably in 7% sodium dodecyl sulfate (SDS), 0.5 M NaPO4, 1 mM EDTA at 50°° C. with washing in 1 X SSC. 0.1% SDS at 50° C. more desirably still in 7% sodium dodecyl sulfate (SDS), 0.5 M NaPO4, 1 mM EDTA at 50°° C. with washing in 0.5 X SSC, 0.1% SDS at 50° C., even more desirably still in 7% sodium dodecyl sulfate (SDS), 0.5 M NaPO4, 1 mM EDTA at 50° C. with washing in 0.1 X SSC, 0.1% SDS at 50° C.

The plants according to the invention may additionally contain an endogenous or a transgene, which confers herbicide resistance, such as the bar or pat gene, which confer resistance to glufosinate ammonium (Liberty®, Basta® or Ignite®) [EP 0 242 236 and EP 0 242246 incorporated by reference]; or any modified EPSPS gene, such as the 2mEPSPS gene from maize [EPO 508 909 and EP 0 507 698 incorporated by reference], or glyphosate acetyltransferase, or glyphosate oxidoreductase, which confer resistance to glyphosate (RoundupReady®), or bromoxynitril nitrilase to confer bromoxynitril tolerance, or any modified AHAS gene. which confers tolerance to sulfonylureas, imidazolinones, sulfonylaminocarbonyltriazolinones, triazolopyrimidines or pyrimidyl (oxy/thio) benzoates, such as oilseed rape imidazolinone-tolerant mutants PM1 and PM2, currently marketed as Clearfield® canola. Further, the plants according to the invention may additionally contain an endogenous or a transgene which confers increased oil content or improved oil composition, such as a 12:0 ACP thioesteraseincrease to obtain high laureate, which confers pollination control, such as such as barnase under control of an anther-specific promoter to obtain male sterility, or barstar under control of an anther-specific promoter to confer restoration of male sterility, or such as the Ogura cytoplasmic male sterility and nuclear restorer of fertility.

The plants and seeds according to the invention may be further treated with a chemical compound, such as a chemical compound selected from the following lists: Herbicides: Clethodim, Clopyralid, Diclofop, Ethametsulfuron, Fluazifop, Glufosinate,

Glyphosate, Metazachlor, Quinmerac. Quizalofop, Tepraloxydim, Trifluralin.

Fungicides 1 PGRs: Azoxystrobin, N-[9-(dichloromethylene)-1,2,3,4-tetrahydro-1,4-methanonaphthalen-5-yl]-3-(difluoromethyl)-1-methyl-1H-pyrazole-4-carboxamide (Benzovindiflupyr, Benzodiflupyr), Bixafen, Boscalid, Carbendazim, Carboxin, Chlormequat-chloride, Coniothryrium minitans, Cyproconazole, Cyprodinil, Difenoconazole, Dimethomorph, Dimoxystrobin, Epoxiconazole, Famoxadone, Fluazinam, Fludioxonil, Fluopicolide, Fluopyram, Fluoxastrobin, Fluquinconazole, Flusilazole, Fluthianil, Flutriafol, Fluxapyroxad, Iprodione, Isopyrazam, Mefenoxam, Mepiquat-chloride, Metalaxyl, Metconazole, Metominostrobin, Paclobutrazole, Penflufen, Penthiopyrad, Picoxystrobin, Prochloraz, Prothioconazole, Pyraclostrobin, Sedaxane, Tebuconazole, Tetraconazole, Thiophanate-methyl, Thiram, Triadimenol, Trifloxystrobin, Bacillus firmus, Bacillus firmus strain I-1582, Bacillus subtilis, Bacillus subtilis strain GB03, Bacillus subtilis strain QST 713, Bacillus pumulis, Bacillus. pumulis strain GB34. Insecticides: Acetamiprid, Aldicarb, Azadirachtin, Carbofuran, Chlorantraniliprole (Rynaxypyr), Clothianidin, Cyantraniliprole (Cyazypyr), (beta-) Cyfluthrin, gamma-Cyhalothrin, lambda-Cyhalothrin, Cypermethrin, Deltamethrin, Dimethoate, Dinetofuran, Ethiprole, Flonicamid, Flubendiamide, Fluensulfone, Fluopyram, Flupyradifurone, tau-Fluvalinate, Imicyafos, Imidacloprid, Metaflumizone, Methiocarb, Pymetrozine, Pyrifluquinazon, Spinetoram, Spinosad, Spirotetramate, Sulfoxaflor, Thiacloprid, Thiamethoxam, 1-(3-chloropyridin-2-yl)-N-[4-cyano-2-methyl-6-(methylcarbamoyl) phenyl]-3- {[5-(trifluoromethyl)-2H-tetrazol-2-yl] methyl}-1H-pyrazole-5-carboxamide, 1-(3-chloropyridin-2-yl)-N-[4-cyano-2-methyl-6-(methylcarbamoyl) phenyl]-3- {[5-(trifluoromethyl)-1H-tetrazol-1-yl] methyl}-1H-pyrazole-5-carboxamide, 1-{2-fluoro-4-methyl-5- [(2.2,2-trifluorethyl) sulfinyl] phenyl}-3-(trifluoromethyl)-1H-1,2,4-triazol-5-amine, (1E)-N- [(6-chloropyridin-3- yl) methyl]-N′-cyano-N-(2,2-difluoroethyl) ethanimidamide, Bacillus firmus, Bacillus firmus strain I-1582, Bacillus subtilis, Bacillus subtilis strain GB03, Bacillus subtilis strain QST 713, Metarhizium anisopliae F52.

Whenever reference to a “plant” or “plants” according to the invention is made, it is understood that also plant parts (cells, tissues or organs, seed pods, seeds, severed parts such as roots, leaves, flowers, pollen, etc.), progeny of the plants which retain the distinguishing characteristics of the parents (especially the fruit dehiscence properties), such as seed obtained by selfing or crossing, e.g. hybrid seed (obtained by crossing two inbred parental lines), hybrid plants and plant parts derived there from are encompassed herein, unless otherwise indicated.

In some embodiments, the plant cells of the invention, i.e. a plant cell comprising a CRT clubroot resistance gene as well as plant cells generated according to the methods of the invention, may be non-propagating cells.

The obtained plants according to the invention can be used in a conventional breeding scheme to produce more plants with the same characteristics or to introduce the characteristic of the presence of the CRT gene according to the invention in other varieties of the same or related plant species, or in hybrid plants. The obtained plants can further be used for creating propagating material. Plants according to the invention can further be used to produce gametes, seeds (including crushed seeds and seed cakes), seed oil, embryos, either zygotic or somatic, progeny or hybrids of plants obtained by methods of the invention. Seeds obtained from the plants according to the invention are also encompassed by the invention.

“Creating propagating material”, as used herein, relates to any means know in the art to produce further plants, plant parts or seeds and includes inter alia vegetative reproduction methods (e.g. air or ground layering, division, (bud) grafting, micropropagation, stolons or runners, storage organs such as bulbs, corms, tubers and rhizomes, striking or cutting, twin-scaling), sexual reproduction (crossing with another plant) and asexual reproduction (e.g. apomixis, somatic hybridization).

As used herein “comprising” is to be interpreted as specifying the presence of the stated features, integers, steps or components as referred to, but does not preclude the presence or addition of one or more features, integers, steps or components, or groups thereof. Thus, e.g., a nucleic acid or protein comprising a sequence of nucleotides or amino acids, may comprise more nucleotides or amino acids than the actually cited ones, i.e., be embedded in a larger nucleic acid or protein. A chimeric gene comprising a nucleic acid which is functionally or structurally defined, may comprise additional DNA regions etc.

The sequence listing contained in the file named “BCS18-2008_ST25.txt ”, which is 74 kilobytes (size as measured in Microsoft Windows®), contains 12 sequences SEQ ID NO: 1through SEQ ID NO: 12 is filed herewith by electronic submission and is incorporated by reference herein.

In the description and examples, reference is made to the following sequences:

### SEQUENCES

SEQ ID No. 1: nucleotide sequence of CRT coding sequence.
SEQ ID No. 2: nucleotide sequence of CRT genomic sequence.
SEQ ID No. 3: amino acid sequence of the CRT protein.
SEQ ID No. 4: nucleotide sequence of CRS1 coding sequence.
SEQ ID No. 5: nucleotide sequence of CRS1 genomic sequence.
SEQ ID No. 6: amino acid sequence of the CRS1 protein.
SEQ ID No. 7: nucleotide sequence of CRS2 coding sequence.
SEQ ID No. 8: nucleotide sequence of CRS2 genomic sequence.
SEQ ID No. 9: amino acid sequence of the CRS2 protein.
SEQ ID No. 10: nucleotide sequence of the T-DNA 35S: : CRT.
SEQ ID No. 11: SNP marker locus associated with the clubroot resistance in the Tosca variety.
SEQ ID No. 12: second SNP marker locus associated with the clubroot resistance in the Tosca variety.

### EXAMPLES

Unless stated otherwise in the Examples, all recombinant DNA techniques are carried out according to standard protocols as described in Sambrook and Russell (2001) Molecular Cloning: A Laboratory Manual, Third Edition, Cold Spring Harbor Laboratory Press, NY, in Volumes 1 and 2 of Ausubel et al. (1994) Current Protocols in Molecular Biology, Current Protocols, USA and in Volumes I and II of Brown (1998) Molecular Biology LabFax, Second Edition, Academic Press (UK). Standard materials and methods for plant molecular work are described in Plant Molecular Biology Labfax (1993) by R.D.D. Croy, jointly published by BIOS Scientific Publications Ltd (UK) and Blackwell Scientific Publications, UK. Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer

Verlag, Germany. Standard procedures for AFLP analysis are described in Vos et al. (1995, NAR 23:4407-4414) and in published EP patent application EP 534858.

### Example 1—Rough Mapping of Clubroot Resistance

### Generation of a Brassica napus Mapping Population

A Double Haploid (DH) population was made from the Fl cross between a clubroot resistant female DH parent and a clubroot susceptible male DH parent. The population consisted of 199 individuals.

### Infinium Genotyping (iSCAN) Analysis of the DH Lines

DNA was extracted from the 199 DH plants and from the 2 parents of the cross using the automated KingFisher DNA extraction method (Thermo Scientific) using the manufacturer's purification kits and protocol.

A custom ˜5K (4921) SNP array, an Illumina Infinium II HD BeadChip was designed by screening Bayer propitiatory SNPs, their individual variant loci including their left and right flanking sequences via the Illumina Assay Design Tool (ADT) then selecting most successful custom genotyping assays as described by Ganal et al. (PLOS. Dec. 8, 2011) and Clark, et al. (2016, Theor Appl Genet. 129 (10): 1887-1899).

Assays were performed as described by the manufacturer's protocol and as described in by Ganal et al. (2011) and Clark, et al. (2016).

Starting with a total of 200 ng (50 ng/μl) of genomic DNA, this is amplified via modified whole genome amplification and enzymically fragmented. The fragmented DNA is hybridized to a single SNP array of a 24 array chip. The position of each oligonucleotide tagged bead is provided in the BeadChip manifest file (*.dmap). In this manner the fragmented DNA sample can hybridise specifically to each bead oligonucleotide which is known to corresponding to a single SNP. Detection of the precise SNP allele is performed by a single base extension, with A/T extension bases being tagged with dinitrophenyl (DNP) and C/G based tagged with biotin. Anti-biotin or anti-DNP antibodies with bound flurophores are lazer exited to emit light signals detected as red and green (respectively) and captured by the Illumina iScan in a high resolution BeadChip image. After images are scanned they are analysed on the Illumina iSCAN system (array reader) where signals are registered according to the corresponding bead map (*.dmap) file and two-colour signal intensity values are extracted for every bead on the image (*.idat). By default, the ICS AutoConvert was enabled, normalising the intensity data and generating genotype calls.

Analysis of BeadArray idat files was performed using GenomeStudioTM Data Analysis Software (GenomeStudio Software V2011.1 (Illumina)). Primary data analyses, such as raw data normalization, clustering, and genotype calling are performed using integrated algorithms (GenTrain & GenCall) in the GenoTyping (GT) Module. Genotype calls are made from BeadChip marker two-colour signal intensities by comparison to canonical genotype clusters. Cluster position identification is performed by the GenTrain algorithm. With one sample precluded due to failed DNA extraction no further poorly performing samples were identified so further sample removal and reclustering of all SNPs was not required. Never-the-less the position of SNP clusters required editing by visualising the Cy3 and Cy5 fluorescence intensity clustering in 2D Cartesian plots and re-centring the clusters. The refined genotype calls were scrutinised further before exporting genotype calls knowing that the parents and the lines of this population were doubled haploids and so any SNPs that failed to show only two-group clustering were not credible: SNPs with more than two call clustered were set to ‘no-call’.

### Phenotyping of DH lines

The University of Alberta, AB Canada, collected isolates across western Canada. Some of the isolates were single-spored and characterized as pathotypes 3, 5, 6 and 8 (Strelkov et. al. 2006, Xue et. 2008) based on the differential system of Williams, 1966. Pathotypes 3, 5, 6and 8 were obtained from the University and each pathotype was individually inoculated onto each DH line separately and phenotyped using a 0-3 scale.

### Genetic Mapping

A total of 1608 polymorphic SNP marker instances were observed over the individuals of the DH mapping population (3070 markers were monomorphic, and 243 had more than 10% no-calls). Genetic linkage mapping was performed using MSTMap software (Wu, PLOS. Oct. 10, 2008). Default MSTMap parameters were applied according to online user instructions (http://138.23.178.42/mstmap/). MSTMap finds the optimal molecular marker order and the recombinational distance by computing a minimal spanning tree of the graph associated with the genotyping data. Setting the minimum distance between two markers, via the parameter ‘no_map_dist’, defined marker dense linkage groups.

Quantitative Trait Locus mapping was performed using R/qtl as described in the

“Guide to QTL Map with R/qtl”. Briefly, the genetic map was inspected by re-calculating the pairwise recombination fractions (recombination frequencies) and the ‘check marker order’ process identified problem marker orders than were then corrected using the ‘switch.order’ function. With the correct orders, final mapping distances were calculated using the Kosambi mapping algorithm. As the phenotypes were not normally distributed we used a rank-based method (an extension of the Kruskal-Wallis test) for ‘Nonparametic interval mapping’

Linked marker genotype groupings were identified by examining the Lod-grouping tree results that ranged from Lod3 to Lod6. With groups of linked markers identified, ordering of the markers was performed using the default calculation options, with the 3rd round option and using the Kosambi mapping function. This analysis led to the identification of a genomic region on chromosome NO3 comprising five candidate disease resistance genes.

### Example 2—Fine mapping of Clubroot Resistance and Identification of the CRT Gene

An F2 population of 3000 individuals was made from the Fl cross between a clubroot resistant female parent and a clubroot susceptible male parent. These 3000 F2 were genotyped together with the parents using 99 SNP markers located in the identified genomic interval on NO3. This analysis led to the identification of a smaller genomic interval within the identified region on chromosome NO3, now comprising only one candidate disease resistance gene, namely the CRT clubroot resistance gene.

The identified CRT gene is a NBS-encoding gene. The genomic sequence of the clubroot resistant allele is provided in SEQ ID NO: 2 while the genomic sequence of two susceptible alleles is provided in SEQ ID Nos: 5 and 8. The corresponding coding DNA sequences are provided respectively in SEQ ID NOs: 1, 4 and 7.

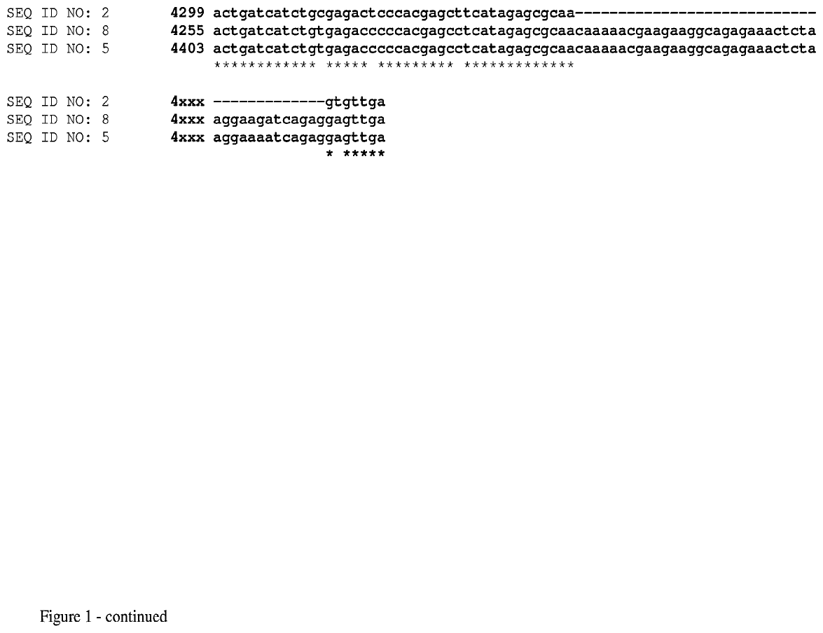

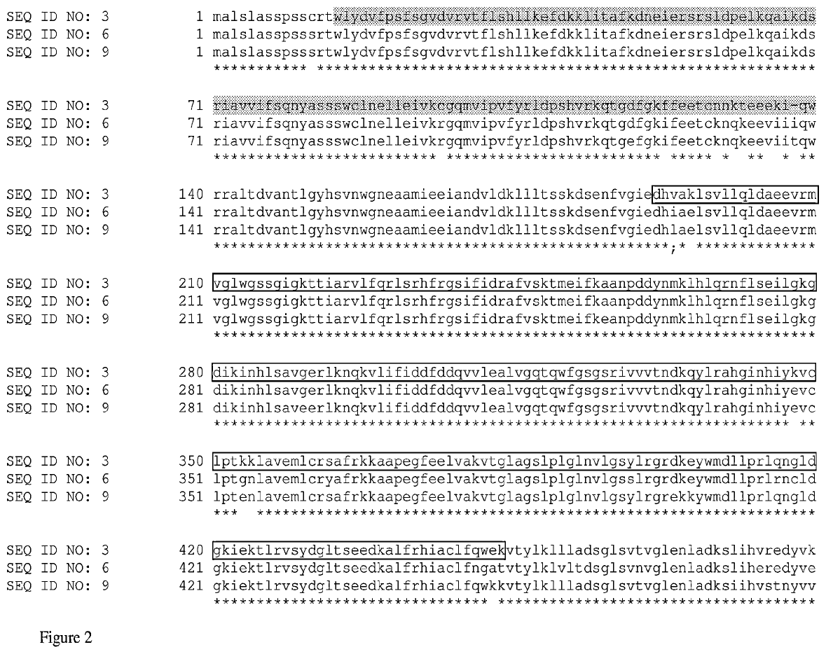

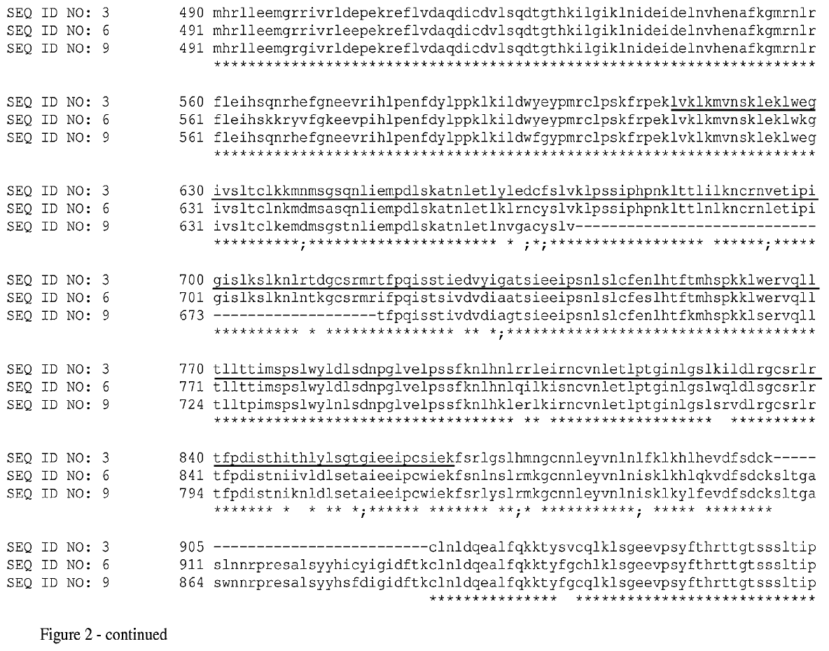

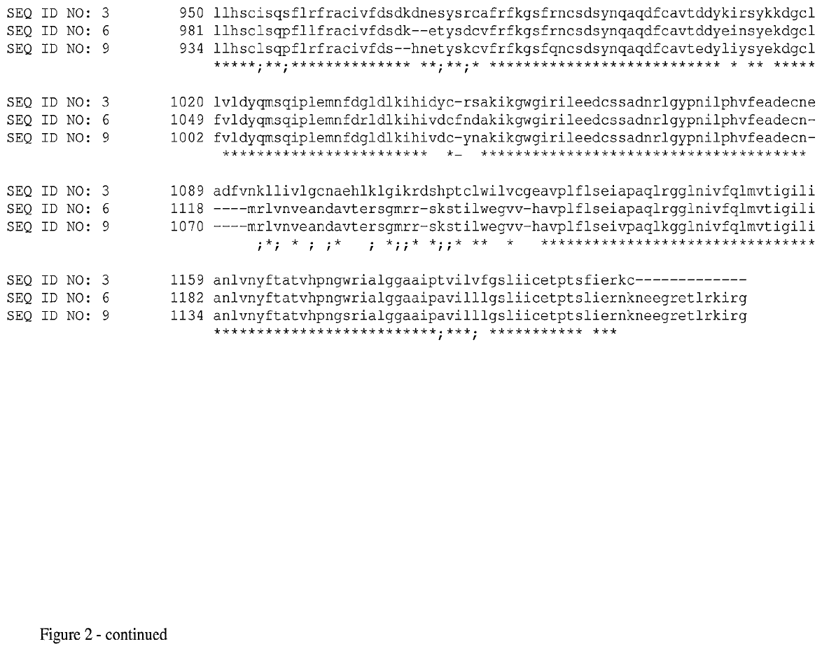

FIG. 1 shows the alignment of the coding sequences of the resistant and susceptible alleles while the alignment of the amino acid sequences of the corresponding resistant and susceptible proteins are given in FIG. 2. Overall, the amino acid sequence of CRT shares at least 90% similarity with the corresponding clubroot sensitive proteins.

### Example 3—Validation of the Clubroot Resistance Gene CRT

### Construction of a Recombinant Gene Encoding CRT for Constitutive Expression in Brassica Cells

A DNA molecule having the nucleic acid sequence according to SEQ ID NO: 1 was obtained by PCR cloning. Using standard recombinant DNA techniques, the constitutive promoter region

CaMV35S according to the sequence from nucleotide position 141 to 668 of SEQ ID NO: 10,the 5′UTR sequence including the leader sequence of the chlorophyll a/b binding protein gene of Petunia hybrid according to the sequence from nucleotide position 672 to 731 of SEQ ID NO: 10, the DNA fragment coding for CRT according to the sequence SEQ ID NO: 1 or to the sequence from nucleotide position 738 to 5084 of SEQ ID NO: 10, and the 3′ untranslated sequence of the g7 gene of Agrobacterium tumefaciens according to the sequence from nucleotide position 5101 to 5304 of SEQ ID NO: 10 were assembled in a vector which contains the bar selectable marker cassette (position 5385 to 7895 of SEQ ID NO: 10) to result in the T-DNA P35S: : CRT (SEQ ID NO: 10).

### Generation of Transgenic Brassica Plants Expressing CRT

The T-DNA vector was introduced into Agrobacterium tumefaciens strains containing a helper Ti-plasmid and used to stably transform an in house developed Brassica napus line which is susceptible to the clubroot strains CR11 and CR6. Homozygous plants and their respective null segregants were further analyzed as described below.

### Clubroot Resistance Assessment of Transgenic Brassica Plants Expressing CRT Constitutively and Their Respective Null Segregating Line

Of all the transgenic lines, or events, produced, 8 were selected based on their effective expression of the CRT transgene in the homozygotes to be phenotyped for Clubroot disease resistance by inoculation with isolate CR6.

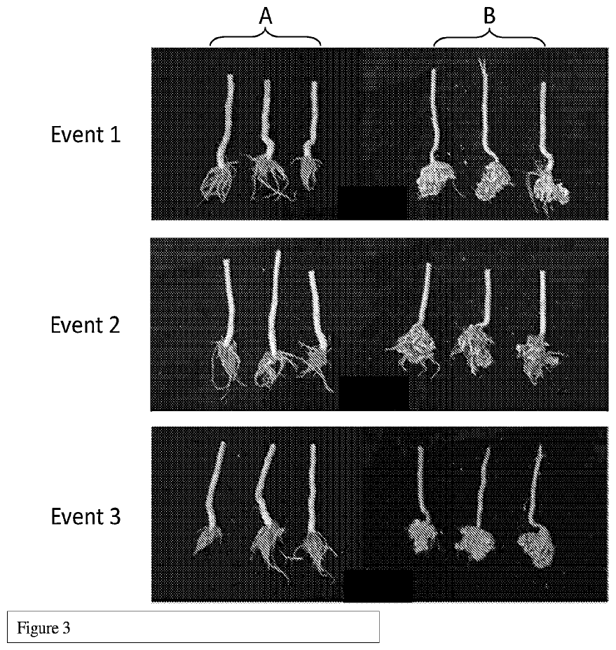

The clubroot resistance assessment provided the consistent result that for each lines transformed with and expressing CRT, the homozygous plants are resistant to the disease while the null segregants are susceptible to it. FIG. 3 shows the phenotype observed for 3 of those lines. The homozygous plants develop healthy root systems in spite of the inoculation with the pathogen while the roots of the null segregants develop galls typical of the infection with the clubroot pathogen.

1. A protein capable of conferring clubroot resistance comprising:
a. an amino acid sequence of SEQ ID NO: 3; or
b. an amino acid sequence having at least 95% sequence identity with the amino acid sequence of SEQ ID NO: 3.

2. An isolated nucleic acid molecule comprising a nucleotide sequence which encodes the protein of claim 1, wherein said nucleic acid is:
a) a nucleic acid comprising a nucleotide sequence of SEQ ID NO: 1 or SEQ ID NO: 2;
b) a nucleic acid sequence having at least 80% identity to the nucleic acid sequence of SEQ ID NO: 1 or SEQ ID NO: 2; or
c) a nucleic acid having a complementary sequence to the nucleic acid of a) or b).

3. A recombinant gene comprising a plant expressible promoter operably linked to a nucleic acid sequence encoding the protein of claim 1 and optionally, a transcription termination and polyadenylation sequence, preferably a transcription termination and polyadenylation region functional in plants.

4. The recombinant gene of claim 3, wherein said nucleic acid is
a. a nucleic acid comprising a nucleotide sequence of SEQ ID NO: 1 or SEQ ID NO: 2;
b. a nucleic acid sequence having at least 80% identity to the nucleic acid sequence of SEQ ID NO: 1 or SEQ ID NO: 2; or
c. a nucleic acid having a complementary sequence to the nucleic acid of a) or b).

5. A plant cell comprising the recombinant gene of claim 3.

6. A plant comprising a heterologous CRT gene encoding the protein according to claim 1 but not comprising at least one of the sequences of SEQ ID NOs: 11 and 12.

7. The plant of claim 6, which is a Brassicaceae.

8. The Brassicaceae plant according to claim 7, further comprising at least one other disease resistance gene, said other disease resistance gene is a clubroot resistance gene, a blackleg resistance gene, a Sclerotinia resistance gene, a Verticillium resistance gene, a Fusarium resistance gene, an Aster Yellows resistance gene, an Alternaria resistance gene, and/or a Grey Stem resistance gene.

9. The Brassicaceae plant according to claim 7, which is Brassica napus, Brassica juncea, Brassica oleracea, Brassica rapa, Brassica nigra or Brassica carinata.

10. Seeds of the plant of claim 7, said seeds comprising said recombinant gene or comprising said heterologous CRT gene but not comprising at least one of the sequences of SEQ ID NOs: 11 and 12.

11. Oil or seed cake from the seed of claim 10.

12. A method of producing food, feed, or an industrial product comprising:
preparing food, feed or industrial product from the plant or part thereof of claim 6.

13. The method of claim 12, wherein
a) the food or feed is oil, meal, grain, starch, flour or protein; or
b) the industrial product is biofuel, fiber, industrial chemicals, a pharmaceutical or a nutraceutical.

14. A kit for the detection of a CRT clubroot resistance locus in Brassica DNA samples, wherein said kit comprises one or more PCR primer pairs, which are able to amplify a DNA marker linked to CRT.

15. A method for determining the presence or absence of CRT in Brassica oilseed plants, comprising amplifying DNA from said Brassica oilseed plants using the one or more PCR primers of the kit of claim 14.
